(19) United States
(12) Patent Application Publication
(10) Pub. No.: US 2012/0148586 A1
(43) Pub. Date: Jun. 14, 2012

(54) GLUCAGON-LIKE PROTEIN-1 RECEPTOR (GLP-1R) AGONISTS FOR TREATING AUTOIMMUNE DISORDERS

(76) Inventors: Joyce Ching Tsu Chou, San Jose, CA (US); Chia-Yang Lin, Palo Alto, CA (US); Hua Long, San Carlos, CA (US)

(21) Appl. No.: 13/392,244

(22) PCT Filed: Aug. 19, 2009
(86) PCT No.: PCT/IB10/53754

§ 371 Date: Feb. 24, 2012

(51) Int. Cl. A61K 39/395; A61P 25/00; A61K 38/00

(52) U.S. Cl. 424/134.1; 424/130.1; 514/17.7

(57) ABSTRACT

Glucagon-like peptide-1 receptor (GLP-1R) agonists are provided for reducing leukocyte invasion of the central nervous system in autoimmune diseases such as multiple sclerosis. GLP-1R agonists include, e.g., naturally-occurring agonists, such as exendin-4, as well as GLP-1R agonist peptides linked to antibodies.

This application claims the benefit of U. S. Provisional Application No. 61/237,654 filed on Aug. 27, 2009, the contents of which is hereby incorporated by reference in its entirety.

### REFERENCE TO SEQUENCES LISTING

This application is being filed electronically and includes an electronically submitted sequence listing in .txt format. The .txt file contains a sequence listing entitled “PC33892ASeqList.txt” created on Aug. 9, 2010 and having a size of 30 KB. The sequence listing contained in this .txt file is part of the specification and is incorporated herein by reference in its entirety.

### FIELD OF THE INVENTION

The invention relates to the treatment of autoimmune diseases affecting the central nervous system, including multiple sclerosis. The invention provides glucagon-like peptide-1 receptor (GLP-1R) agonists for reducing leukocyte invasion of central nervous system tissues.

### BACKGROUND

Multiple sclerosis (MS) is a demyelinating autoimmune disease of the central nervous system (CNS) characterized by inflammation, demyelination, and axonal injury. The disease affects more than a million people world-wide and is twice as prevalent in women than in men. The symptoms of MS usually appear between age 20 and 40.

The etiology of MS is unclear, although features of the disease have been studied. Such features include damage to CNS tissues, activation of microglia, proinflammatory cytokine production, arrest of T cell migration and clonotypic expansion, altered macrophage effector function, production, upregulation of MHC, and direct CNS attack by infiltrating T cells.

Experimental autoimmune encephalomyelitis (EAE) is considered a standard model for MS. The murine disease model has been used to study the etiology of MS and to evaluate drugs for its treatment (Aharoni, R. et al., 2005, J. Neurosci. 25:8217-28). The clinical features of EAE include inflammation and demyelination of the CNS by large numbers of infiltrating lymphocytes and macrophages. Active immunization of mice with several different protein components of myelin, including myelin basic protein (MBP), proteolipid protein (PLP), and myelin oligodendrocyte glycoprotein (MOG), induces the production of autoimmune antibodies and the clinical symptoms of ascending paralysis. The disease may be acute or chronic depending on the mouse strain and the myelin protein used for immunization. EAE has been used to study the etiology of MS and to evaluate drugs for its treatment (Aharoni, R. et al.).

MS results largely from a T cell response to myelin, including lipids and several proteins abundant in neuronal tissues. Demyelination and clinical paralysis result from the invasion of CNS tissues by T cells of the Th1 and Th17 phenotypes, with specificity for myelin antigens. Th1 cells produce inflammatory cytokines including TNF-α and IFN-γ. IL-17 expression is increased in patients with MS, as well as patients with rheumatoid arthritis and irritable bowel disease. (Komiyama, Y. et al., 2006, J. Immunol. 177, 566-573; Matusevicius, D. et al., 1999, Mult. Scler. 5, 101-104. This suggests a pathogenic role for Th17 cells in autoimmune disease. Damage to the CNS is likely also caused by other immunological responses, including the production of autoimmune antibodies and complement activation. B-cells are involved during early and late stages of MS and EAE with MBP-specific and MOG-specific antibodies of various isotypes found throughout the courses of the diseases. Sections prepared from brain and spinal cord tissues show leukocyte invasion (particularly lymphocytes and macrophages) and destruction of the underlying tissues of the nervous system).

Glucagon-like peptide (7-36) amide (GLP-1) is a glucoincretin both in rat and in man (Dupre and Ebert and Creutzfeld, 1987, Diabetes Metab. Rev. 3; Mojsov et al., 1986, J. Biol. Chem. 261:11880). In addition to increasing insulin secretion and decreasing glucagon secretion, the 30-amino acid GLP-1 peptide stimulates pro-insulin gene transcription, slows down gastric emptying time, and reduces food intake. GLP-1 exerts its physiological effects by binding to the glucagon-like peptide 1 receptor (GLP-1R). GLP-1R belongs to the class B receptor sub-class of the G protein-coupled receptor (GPCR) superfamily that regulates many important physiological and pathophysiological processes. GLP-1R is a seven-transmembrane spanning receptor coupled to G-protein activation, increased cAMP production and activation of PKA. There are also PKA-independent responses initiated through the GLP-1R. Other responses to the actions of GLP-1 include, for example, pancreatic β-cell proliferation and expansion concomitant with a reduction of β-cell apoptosis. In addition, GLP-1 activity can result in increased expression of the glucose transporter-2 (GLUT2) and glucokinase genes in pancreatic cells.

GLP-1 is also a neuropeptide synthesized by neurons in the caudal regions of the nucleus of the solitary tract (NTS) (Jin, S. L. et al., 1988, J Comp Neurol. 271:519-532). GLP-1-immunoreactive fibers and GLP-1Rs appear to be expressed widely throughout the brain (Jin, S. L. et al., 1988; Larsen, P. J. et al., 1997, Neuroscience 77:257-270; Merchenthaler, I. et al., 1999, J Comp Neurol 403:261-280). GLP-1R-deficient mice have enhanced seizure severity and neuronal injury after kainate administration, with phenotypic correction after Glp1r gene transfer in hippocampal somatic cells (During, M. J. et al., 2003, Nat Med. 9(9):1173-9).

### SUMMARY

In one aspect, the invention provides a method for reducing leukocyte invasion of a tissue of the central nervous system comprising administering to a mammal in need of such treatment a composition comprising a GLP-1R agonist in an amount effective for activating GLP-1R, thereby reducing leukocyte invasion of a tissue of the central nervous system. The present invention further provides the use of a GLP-1R agonist in the manufacture of a medicament used for reducing leukocyte invasion of a tissue of the central nervous system in a mammal. The present invention further provides the use of a GLP-1R agonist in the manufacture of a medicament used for reducing Th1 and Th17 cell numbers in a mammal.

In some embodiments, the mammal has an autoimmune disorder. In one embodiment, the autoimmune disorder is experimental autoimmune encephalomyelitis. In another embodiment, the autoimmune disorder is multiple sclerosis. In other embodiments, the autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, or Graves disease. In preferred embodiments, the mammal is a human.

In some embodiments, administration of the composition comprising the GLP-1R agonist reduces the number of Th1 and/or Th17 cells in a lymph node of the mammal. In some embodiments, administration of the composition comprising the GLP-1R agonist reduces the Ter119+ erythroid cells in a lymph node of the mammal.

In preferred embodiments, the invading leukocytes comprise T cells and macrophages. In particular embodiments, the invading leukocytes comprise CD3-expressing and CD68-expressing leukocytes. In preferred embodiments, the tissue of the central nervous system is brain tissue or spinal cord tissue.

In some embodiments, the GLP-1R agonist is OAP-189. In other embodiments, the GLP-1R agonist is a DPP-4 inhibitor. In other embodiments, the GLP-1R agonist is an anti-GLP-1R agonist antibody.

In some embodiments, the GLP-1R agonist is a naturally-occurring GLP-1R agonist. In some embodiments, the naturally-occurring GLP-1R agonist is exendin-4. In related embodiments, the naturally-occurring GLP-1R agonist comprises a fragment or derivative of exendin-4. In some embodiments, the fragment or derivative of exendin-4 binds to and activates GLP-1R.

In some embodiments, the GLP-1R agonist comprises a GLP-1R agonist Antibody Conjugate (“GAC”) comprising a GLP-1R agonist peptide and an antibody. In related embodiments, the GAC can be of the structure:

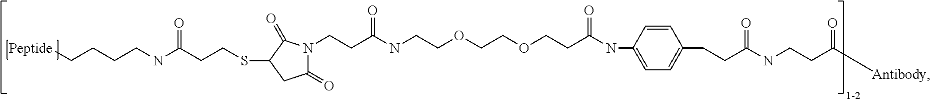

wherein the peptide is of the formula: R¹—[H¹X²E³G⁴T⁵F⁶T⁷S⁸D⁹X¹⁰S¹¹X¹²X¹³X¹⁴E¹⁵ X¹⁶X¹⁷A¹⁸X¹⁹X²⁰X²¹F²²X²³X²⁴X²⁵X²⁶X²⁷X²⁸X²⁹X³⁰X³¹X³²X³³X³⁴X³⁵X³⁶X³⁷X³⁸ X³⁹ X⁴⁰ (SEQ ID NO: 3)]-R², wherein R¹ is absent, CH₃, C(O)CH₃, C(O)CH₂CH₃, C(O)CH₂CH₂CH₃, or C(O)CH(CH₃)CH₃; R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate, and X² is a blocking group such as Aib, A, S, T, V, L, I, D-Ala; X¹⁰ is V, L, I, or A; X¹² is S or K; X¹³ is Q or Y; X¹⁴ is G, C, F, Y, W, M, or L; X¹⁶ is K, D, E, or G; X¹⁷ is E or Q; X¹⁹ is L, I, V, or A; X²⁰ is ornithine or a derivatized lysine group such as K(SH) R, or K; X²¹ is L or E; X²³ is I or L; X²⁴ is A or E; X²⁵ is W or F; X²⁶ is L or I; X²⁷ is I, K, or V; X²⁸ is R, ornithine, N, or K; X²⁹ is Aib or G; X³⁰ is any amino acid, preferably G or R; X³¹ is P or absent; X³² is S or absent; X³³ is S or absent; X³⁴ is G or absent; X³⁵ is A or absent; X³⁶ is P or absent; X³⁷ is P or absent; X³⁸ is P or absent; X³⁹ is S or absent; and X⁴° is a linking residue or absent; and wherein one of X¹⁰, S¹¹, X¹², X¹³, X¹⁴, X¹⁶, X¹⁷, X¹⁹, X²⁰, X²¹, X²⁴, X²⁶, X²⁷, X²⁸, X³², X³³, X³⁴, X³⁵, X³⁶, X³⁷, X³⁸, X³⁹, or X⁴⁰ is substituted with a linking residue comprising a nucleophilic side chain covalently linked to the combining site of the antibody via a linker, wherein the linking residue is selected from the group consisting of K, R, Y, C, T, S, homologs of lysine (including K(SH)), homocysteine, and homoserine.

In some embodiments, the GAC can comprise the structure:

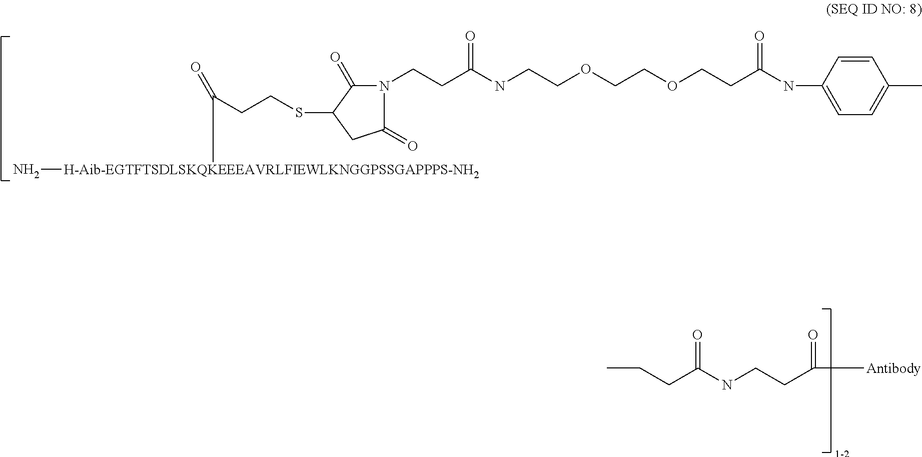

In some embodiments, the GAC can comprise the structure:

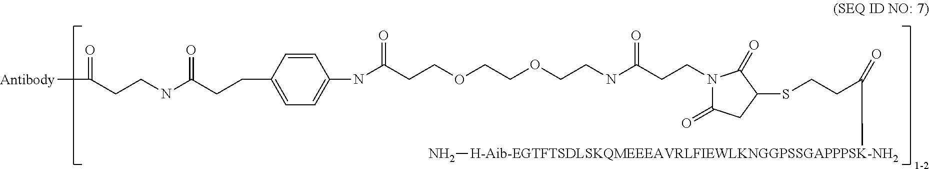

In some embodiments, the antibody is selected from the group consisting of a full length antibody, a Fab, a Fab′, a F(ab′)2, an Fv, a dsFv, an scFv, a VH, a diabody and a minibody. In some embodiments, the antibody comprises a constant domain selected from the group consisting of IgG1, IgG2, IgG3, and IgG4. In some embodiments, the antibody is a catalytic antibody. In some embodiments, the antibody is an aldolase antibody. In some embodiments, the aldolase antibody comprises a light chain comprising the amino acid sequence set forth in SEQ ID NO: 11 and a heavy chain amino acid sequence comprising the amino acid sequence set forth in SEQ ID NO: 12.

In other embodiments, the invention provides a kit of parts for reducing leukocyte invasion of a tissue of the central nervous system, comprising a GLP-1R agonist in an amount effective for activating GLP-1R, and instructions for use. In related embodiments, the invention provides a kit of parts for treating an autoimmune disorder affecting the central nervous system, comprising a GLP-1R agonist in an amount effective for activating GLP-1R and instructions for use.

These and other aspects of the invention will become apparent from the description and examples that follow.

### BRIEF DESCRIPTION OF THE DRAWINGS

FIG. 1 depicts histochemistry showing infiltrating cell bodies, T cells, monocytes and microglia in EAE animals after MOG immunization. Spinal cord sections from EAE animals (top panel) and healthy animals (bottom panel) were stained with Cresyl violet to stain infiltrating cell bodies (left), anti-CD3 antibodies to detect infiltrating T cells (middle), or anti-CD68 antibodies to detect monocytes and microglia (right).

FIG. 2 depicts a graph showing clinical severity of EAE in animals treated with 1 mg/kg GLP-1R agonist following MOG immunization. Animals received daily, from day 0 to day 9 after MOG immunization, either 1 mg/kg exendin-4 (EXE4), 2 mg/kg neurotrophin-4 (NT4), or PBS (control). Mice were assessed daily for clinical signs of EAE according to the following scoring system: 0=normal; 1=limp tail; 2=moderate hind limb weakness; 3=moderately severe hind limb weakness (animal can still walk with difficulty); 4=severe hind limb weakness (animal can move its hind limbs but cannot walk); 5=complete hind limb paralysis; and 6=death.

FIG. 3 depicts a graph showing morbidity in EAE animals treated with 1 mg/kg of GLP-1R agonist following MOG immunization. Animals were treated either daily, from day 0 to day 6 after MOG immunization, with 1 mg/kg exendin-4 (EXE4) or PBS, or weekly with the GLP-1R agonist GAC-1 (1 mg/kg). Clinical scores were monitored daily and were assessed as described above.

FIG. 4 depicts a graph showing morbidity in EAE animals treated with varying does of GLP-1R agonist (exendin-4 or GAC-1) following MOG immunization. The exendin-4 (EXE4) treated animals were treated daily from day 0 to day 2 after MOG immunization with 3mg/kg exendin-4, and daily from day 3 to day 8 with 1 mg/kg exendin-4. The GAC-1 treated animals were treated with 3 mg/kg GAC-1 on day 0, followed by weekly treatments of 1 mg/kg GAC-1 starting on day 7 after immunization. Control animals were treated daily with control PBS. Clinical scores were monitored daily. Clinical signs of EAE were assessed as described above.

FIGS. 5A-C depicts the results of histochemical staining of spinal cord sections from (A) PBS control treated and (B) exendin-4-treated and (C) GAC-1-treated EAE animals. The sections were stained with Luxol Fast Blue for myelin. Black arrows indicate demyelination in the white matter showing reduced Luxol Fast Blue staining.

FIGS. 6A-F depict the results of immunohistochemical staining of spinal cord sections from PBS control (A, D) and exendin-4-treated (B, E) and GAC-1-treated (C, F) EAE animals. The sections were stained with an antibody specific for CD3 (A-C) or CD68 (D-F). Sections shown are the ventral horn of the spinal cord cross section.

FIG. 7 depicts graphs showing data from flow cytometry analysis of CD11b+CD45^{hi} cell (activated microglia and infiltrating macrophages) and CD11b+CD45^{lo} cell (resting microglia) populations from brain and spinal cord stained with antibodies specific for MHC class II. In each graph, MHC class II expression of cells from exendin-4 (EXE4) treated EAE animals is indicated by the black line without fill (□), and MHC class II expression of cells from control treated EAE animals is indicated by the dotted line ( [symbol] ) Top panel: cells collected from animals at disease onset. Bottom panel: cells collected from animals at disease peak.

FIG. 8 depicts graphs showing pathogenicity of splenocytes in an adoptive transfer model of EAE. Left graph: animals received daily from day 0 to day 4 after PLPp(139-151) immunization either 1 mg/kg exendin-4 (EXE4) or PBS (control).

Animals were sacrificed on day 5 after immunization. Right graph: splenocytes from exendin-4 treated donors (triangles) and PBS treated donors (squares) were transferred to naïve SJL/J animals. Recipient mice were monitored daily for clinical score and body weight changes. Clinical signs of EAE were assessed as described above.

FIGS. 9A-C depict lymph node weight and spleen weight and size after treatment with a GLP-1R agonist. Animals were treated daily from day 0 to day 5 after MOG immunization with 1 mg/kg exendin-4 (EAE-EXE4), PBS (EAE-vehicle) or 4 mg/kg dexamethasone control (EAE-Dex). Naïve animals were not immunized with MOG. (A) Inguinal lymph node weight. (B) Spleen weight. (C) Spleens from a naïve animal, EAE animal treated with vehicle and an EAE animal treated with exendin-4.

FIGS. 10A-F depict graphs showing immunological changes in EAE animals after treatment with a GLP-1R agonist following MOG immunization. Animals were treated with 1 mg/kg exendin-4 (EXE4) or PBS daily from day 0 to day 6 after MOG immunization. Animals were sacrificed on day 6 after immunization, together with mice that were not immunized (naïve). Lymphocytes (A-C) and monocytes (D-F) were measured from spleen (A, D), lymph nodes (B, E) and peripheral blood (C, F). The y-axis indicates the percentage of cells that are lymphocytes (A-C) or monocytes (D-F).

FIGS. 11A-F depict graphs showing immunological changes in EAE animals after treatment with a GLP-1R agonist following MOG immunization. Animals were treated with 1mg/kg exendin-4 (EXE4) or PBS daily from day 0 to day 6 after MOG immunization. Animals were sacrificed on day 6 after immunization, together with mice that were not immunized (naive). CD4+ cells (A-C) and CD8+ cells (D-F) were measured from spleen (A, D), lymph nodes (B, E) and peripheral blood (C, F). The percentage of cells staining positive for the indicated marker is shown on the y-axis.

FIGS. 12A-F depict graphs showing immunological changes in EAE animals after treatment with a GLP-1R agonist following MOG immunization. Animals were treated with 1 mg/kg exendin-4 (EXE4) or PBS daily from day 0 to day 6 after MOG immunization. Animals were sacrificed on day 6 after immunization, together with mice that were not immunized (naïve). CD19+ cells (A-C) and Ter119+ cells (D-F) were measured from spleen (A, D), lymph nodes (B, E) and peripheral blood (C, F). The percentage of cells staining positive for the indicated marker is shown on the y-axis.

FIG. 13 depicts a graph showing Ter119+ cell population change in EAE animals after treatment with a GLP-1R agonist following MOG immunization. Animals were treated with PBS (EAE-PBS), 1 mg/kg exendin-4 (EAE-EXE4) or 4 mg/kg dexamethasone (EAE-dex) daily from day 0 to day 5. Animals were sacrificed on day 5 after immunization, together with mice that were not immunized (naïve). Spleen cells were surfaced stained for erythroid lineage marker Ter119. The percentage of cells staining positive for Ter119 is indicated on the y-axis.

FIG. 14 depicts a graph showing activated T cell population change in EAE animals after treatment with a GLP-1R agonist following MOG immunization. Animals were treated with PBS (EAE-PBS), 1mg/kg exendin-4 (EAE-EXE4) or 4 mg/kg dexamethasone (EAE-dex) daily from day 0 to day 5. Animals were sacrificed on day 5 after immunization, together with mice that were not immunized (naïve). Activated T cells from lymph node were identified as CD4+ cells having high levels of CD44 (CD44^{hi}). The y-axis indicates the percentage of CD4+ cells having high levels of CD44 in each treatment group and the naïve group.

FIG. 15 depicts a graph showing proliferating cell population change in EAE animals after treatment with a GLP-1R agonist following MOG immunization. Animals were treated with PBS (EAE-PBS), 1 mg/kg exendin-4 (EAE-EXE4) or 4 mg/kg dexamethasone (EAE-dex) daily from day 0 to day 5. Animals were sacrificed on day 5 after immunization, together with mice that were not immunized (naïve). Lymph node cells were cultured with MOG stimulation and treated with BrdU to measure cell proliferation. The y-axis indicates the percentage of CD4+ that were BrdU positive in each treatment group and the naïve group.

FIG. 16 depicts graphs showing IL-17+ cell population change in EAE animals after treatment with a GLP-1R agonist following MOG immunization. Animals were treated with PBS (EAE-PBS), 1 mg/kg exendin-4 (EAE-EXE4) or 4 mg/kg dexamethasone (EAE-dex) daily. Animals were sacrificed on day 5 (left graph) or day 7 (right graph) after immunization, together with mice that were not immunized (naïve). Inguinal lymph node cells were stained for CD4, IL-17 and IFN-γ. The percentage of CD4+ cells staining positive for IL-17 is indicated on the y-axis.

FIG. 17 depicts a graph showing interferon-γ (IFN-γ)+ cell population change in EAE animals after treatment with a GLP-1R agonist following MOG immunization. Animals were treated with PBS (EAE-PBS), 1 mg/kg exendin-4 (EAE-EXE4) or 4 mg/kg dexamethasone (EAE-dex) daily from day 0 to day 5. Animals were sacrificed on day 5 after immunization, together with mice that were not immunized (naïve). Inguinal lymph node cells were stained for CD4, IL-17 and IFN-γ. The percentage of CD4+ cells staining positive for IFN-γ is indicated on the y-axis.

FIG. 18 depicts a graph showing morbidity in EAE animals treated with GLP-1R agonist (exendin-4) or DPP-4 inhibitor (sitagliptin) following MOG immunization. MOG immunized animals were dosed with 1 mg/kg sitagliptin, 10 mg/kg sitagliptin, exendin-4, or methylcellulose. Clinical scores were monitored daily. Clinical signs of EAE were assessed as described above.

FIG. 19 depicts a graph showing morbidity in EAE animals treated with GLP-1R agonist (exendin-4) or vehicle. Beginning at day 29 after immunization, PLP immunized SJL/J mice were dosed daily with 1 mg/kg exendin-4 or vehicle. Clinical scores were monitored daily. Clinical signs of EAE were assessed as follows: 0, no paralysis; 1, loss of tail tone; 2, hindlimb weakness; 3, hindlimb paralysis; 4, hindlimb and forelimb paralysis; 5, moribund or dead.

FIG. 20 depicts a graph showing morbidity in vagotomized and non-vagotomized EAE animals treated with exendin-4 or PBS. Mice were assessed daily for clinical signs of EAE according to the following scoring system: 0=normal; 1=limp tail; 2=moderate hind limb weakness; 3=moderately severe hind limb weakness (animal can still walk with difficulty); 4=severe hind limb weakness (animal can move its hind limbs but cannot walk); 5=complete hind limb paralysis; and 6=death.

### DETAILED DESCRIPTION

The present invention relates to methods of using GLP-1R agonists for the treatment of multiple sclerosis and other autoimmune disorders.

### General Techniques

The present invention employs conventional techniques used in the fields of molecular biology, cell biology, biochemistry and immunology. Such techniques are described in references, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following abbreviations, terms and phrases are used herein as defined below. Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular.

| | One letter | Three letter |
|---|---|---|
| Amino acid | abbreviation | abbreviation |
| 2-aminoisobutyric | — | Aib2 or Aib |
| acid | | |
| Alanine | A | Ala |
| Arginine | R | Arg |
| Asparagine | N | Asn |
| Aspartic acid | D | Asp |
| Cysteine | C | Cys |
| Glutamic acid | E | Glu |
| Glutamine | Q | Gln |
| Glycine | G | Gly |
| Histidine | H | His |
| Isoleucine | I | Ile |
| Leucine | L | Leu |
| Lysine | K | Lys |
| Methionine | M | Met |
| Norleucine | — | Nle |
| Ornithine | — | Orn |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless indicated otherwise by a “D” prefix, e.g., D-Ala or N-Me-D-Ile, the stereochemistry of the alpha-carbon of the amino acids and aminoacyl residues in peptides described herein is the natural or “L” configuration. The Cahn-Ingold-Prelog “R” and “S” designations are used to specify the stereochemistry of chiral centers in certain acyl substituents at the N-terminus of the peptides. The designation “R,S” is meant to indicate a racemic mixture of the two enantiomeric forms. This nomenclature follows that described in R. S. Cahn, et al., Angew. Chem. Int. Ed. Engl., 5:385-415 (1966).

D-H refers to D Histidine.

2-aminoisobutyric acid as used herein has the following structure:

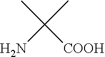

“Polypeptide,” “peptide,” and “protein” are used interchangeably to refer to a polymer of amino acid residues. As used herein, these terms may apply to amino acid polymers in which one or more amino acid residues is an artificial chemical analog of a corresponding naturally occurring amino acid. These terms also apply to naturally occurring amino acid polymers. Amino acids can be in the L or D form as long as the binding function of the peptide is maintained. Peptides may be cyclic, having an intramolecular bond between two non-adjacent amino acids within the peptide, e.g., backbone to backbone, side-chain to backbone and side-chain to side-chain cyclization. Cyclic peptides can be prepared by methods well know in the art. See, e.g., U.S. Pat. No. 6,013,625; S. Cheng et al., J Med. Chem. 37:1-8 (1994).

All peptide sequences are written according to the generally accepted convention whereby the alpha-N-terminal amino acid residue is on the left and the alpha-C-terminal amino acid residue is on the right. As used herein, the term “N-terminus” refers to the free alpha-amino group of an amino acid in a peptide, and the term “C-terminus” refers to the free carboxylic acid terminus of an amino acid in a peptide. A peptide which is N-terminated with a group refers to a peptide bearing a group on the alpha-amino nitrogen of the N-terminal amino acid residue. An amino acid which is N-terminated with a group refers to an amino acid bearing a group on the alpha-amino nitrogen.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (scFv) and domain antibodies (including, e.g., shark and camelid antibodies), and fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “invading leukocytes” are leukocytes that invade, infiltrate, or migrate into tissues of the central nervous system (CNS), including brain and spinal cord tissues, as a result of an autoimmune disease, preferably an autoimmune disease affecting the CNS. The invading leukocytes are primarily T cells and monocytes, although other leukocytes may be present.

As used herein, “reducing leukocyte invasion” refers to decreasing the migration (i.e., invasion or infiltration) of leukocytes into tissues of the CNS, including brain and spinal cord tissues. Reducing leukocyte invasion also refers to reducing the cytotoxic affects mediated by leukocyte invasion, particularly with respect to the underlying neuronal cells and/or other supporting cells of the CNS tissue. Leukocyte invasion includes invasion by T cells and monocytes. Reducing leukocyte invasion includes protecting CNS tissues from autoimmune attack. The cells of the CNS that are destroyed by leukocyte invasion include myelin-expressing cells and neighboring non-myelin expressing cells. Cell destruction may be by apoptosis, necrosis, or a combination, thereof. Reduced leukocyte invasion is characterized by such clinical indications as slowed disease progression, delayed onset or severity of morbidity, prolonged survival, improved quality of life, decreased or stabilized cognitive, motor, or behavioral symptoms. Reducing leukocyte invasion also includes preventing or reducing the risk of migration of leukocytes into tissues of the central nervous system (CNS). The reducing in leukocyte invasion may be partial or complete, for example, the reduction may be about 10%, about 20%, about 30%, about 40%, about 50%, about 60%, about 70%, about 80%, about 90%, or even about 95%, in comparative or actual reduction, as described herein.

As used herein, the term “GLP-1R” refers to any form of GLP-1R and variants thereof that retain at least part of the activity of GLP-1R. Unless indicated differently, such as by specific reference to human GLP-1R, GLP-1R includes all mammalian species of native sequence GLP-1R, e.g., human, canine, feline, equine, and bovine.

As used herein, a “GLP-1 receptor agonist” or a “GLP-1R agonist” is a molecule that increases the amount of activation of GLP-1R, producing effects similar to those produced by the naturally-occurring agonists GLP-1 and exendin-4. GLP-1R agonists increase the activation of GLP-1R, e.g., by binding to and activating GLP-1R, by causing a conformational change in the GLP-1R, by causing activation of a G protein coupled to the GLP-1R, by causing GLP-1R to remain in an activated (e.g., in the active conformation) condition for a longer period of time (including indefinitely), by mimicking the binding of naturally-occurring agonists, by modulating the binding of naturally-occurring agonists, by blocking inhibitors of GLP-1 or otherwise modulating GLP-1R activation or initiating the cascade of intracellular events that is characteristic of GLP-1R activation. Preferred properties of GLP-1R agonist are described herein. GLP-1R agonists of the invention may increase activation of GLP-1R by at least 5%, at least 10%, at least 20%, at least 30%, at least 50%, at least 100%, at least 200%, or more.

As used herein, “naturally-occurring GLP-1R agonists” are molecules that exist in nature and function as activators of GLP-1R. The known naturally-occurring agonists of GLP-1R are the peptide hormones GLP-1 and exendin-4. Naturally-occurring GLP-1R agonists include naturally-occurring variant molecules, such as a polypeptide expressed in an animal with a mutated GLP-1R allele.

“Biological activity”, when used in conjunction with the GLP-1R agonist of the present invention, generally refers to having the ability to bind and activate GLP-1R and/or a downstream pathway mediated by the GLP-1R signaling function. As used herein, “biological activity” encompasses one or more effector functions in common with those induced by action of GLP-1, the native ligand of GLP-1R, on a GLP-1R-expressing cell. Without limitation, biological activities include any one or more of the following: the ability to bind and activate GLP-1R; binding to and activating GLP-1R, the ability to cause a conformational change in the GLP-1R, the ability to cause activation of a G protein coupled to the GLP-1R, the ability to cause GLP-1R to remain in an activated (e.g., in the active conformation) condition for a longer period of time (including indefinitely), the ability to increase intracellular cAMP, the ability to stimulate insulin release, and the ability to initiate the cascade of intracellular events that is characteristic of GLP-1R activation.

As used herein a “full agonist” is an agonist which, at an effective concentration, essentially completely induces a measurable effect of GLP-1R. For example, the measurable effect of GLP-1R may be increased cAMP levels. By a partial agonist is meant an agonist that is capable of partially inducing a measurable effect, but that, even at a highest concentration is not a full agonist. By essentially completely is meant at least about 80%, preferably, at least about 90%, more preferably, at least about 95%, and most preferably, at least about 98% of the measurable effect is induced. The relevant “measurable effects” are described herein.

As used herein, “a fragment” polypeptide is a portion of a larger polypeptide that retains at least some of the biological properties or the larger polypeptide, such as the ability to activate GLP-1R. Preferred fragments comprise the amino acid residues and/or structures or the larger polypeptide that resulted in the biological properties of the larger polypeptide. Polypeptide fragments may be called peptide, although no distinction is made herein between polypeptides and peptide. Exemplary fragments are described herein. Fragments may be derivativized as described herein.

As used herein, “a derivative” polypeptide has one or more covalent or non-covalent modifications, such as the addition or removal of a functional group or moiety. Examples of derivatives are provided herein.

As used herein, a “variant” of a particular polypeptide sequence is defined as a polypeptide sequence having at least 40% sequence identity to the particular polypeptide sequence over a certain length of one of the polypeptide sequences using an algorithm such as Clustal V or BLAST, e.g., the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

As used herein, “sequence identity,” as applied to polypeptide sequences, refer to the percentage of residue matches between at least two polypeptide sequences aligned using a standardized algorithm, such as Clustal V, MEGALIGN, or BLAST. Methods of polypeptide sequence alignment are well-known. Some alignment methods take into account conservative amino acid substitutions. Such conservative substitutions as described herein, and generally preserve the charge and hydrophobicity at the site of substitution, thus preserving the structure (and therefore function) of the polypeptide.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR', CO or CH₂ (“formacetal”), in which each R or R′ is independently H or substituted or unsubstituted alkyl (1-20° C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “treatment” is an approach for obtaining beneficial or desired clinical results. For purposes of this invention, beneficial or desired clinical results include, but are not limited to, one or more of the following: improving mobility, reducing limb weakness, reducing leukocyte invasion of the CNS, and reducing demyelination resulting from autoimmune diseases, including MS.

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the GLP-1R agonist based on a reasonable expectation that such administration may likely cause such a reduction in incidence in that particular individual.

“Ameliorating” means a lessening or improvement of one or more symptoms as compared to not administering a GLP-1R agonist. “Ameliorating” also includes shortening or reduction in duration of a symptom.

As used herein, “an amount effective for activating GLP-1R,” or similar expressions with respect to a GLP-1R agonist, refers to a quantity sufficient to increase in GLP-1R activation (as defined herein and known in the art) compared to a baseline level of activation prior to the administration of GLP-1R agonist. The increase in activation may be at least 5%, at least 10%, at least 20%, at least 30%, at least 50%, at least 100%, at least 200%, or more. This amount will take into account such considerations as the route of administration, the half-life of the GLP-1R agonist in the body, the solubility, bioavailability, clearance rate, and other pharmacokinetic characteristics of the GLP-1R agonist, the body weight and metabolism of the animal or patient, etc.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as, for example, reducing one or more symptoms of CMT disease, decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. As used herein, “an animal in need of treatment,” or similar expressions, means an animal, preferably a mammal including a human, having or at risk for developing an autoimmune disease involving the CNS. Examples of autoimmune diseases (or disorders, without distinction) affecting the CNS are experimental autoimmune encephalomyelitis (EAE) in mice, multiple sclerosis (MS) in humans, and similar autoimmune diseases found in other mammals. Autoimmune attack of the CNS is also observed in, e.g., immune rejection, optic neuropathies, inflammatory bowel disease, and Parkinson's disease.

An “individual” or a “subject” is a mammal, more preferably, a human. Mammals also include, but are not limited to, farm animals, sport animals, pets, primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

As used herein, “peripheral administration” or “administered peripherally” refers to introducing an agent into a subject outside of the central nervous system (CNS) or blood brain barrier (BBB). Peripheral administration encompasses any route of administration other than direct administration to the spine or brain. Peripheral administration can be local or systemic.

### GLP-1R Agonists for the Treatment of Autoimmune Disorders Affecting the Central Nervous System (CNS)

The invention provides methods of using GLP-1R agonists for the treatment of multiple sclerosis and other autoimmune disorders affecting the central nervous system (CNS). According to one feature of the invention, GLP-1R agonists are effective in reducing leukocyte invasion into tissues of the CNS and reducing the destruction of the underlying neuronal tissues of the CNS, and thus ameliorating the clinical manifestations of this disease, such as limb paralysis. In particular, GLP-1R agonists reduce the migration of monocytes and T cells, which are active in presenting myelin antigens and administering cytotoxic effects on the cells producing them.

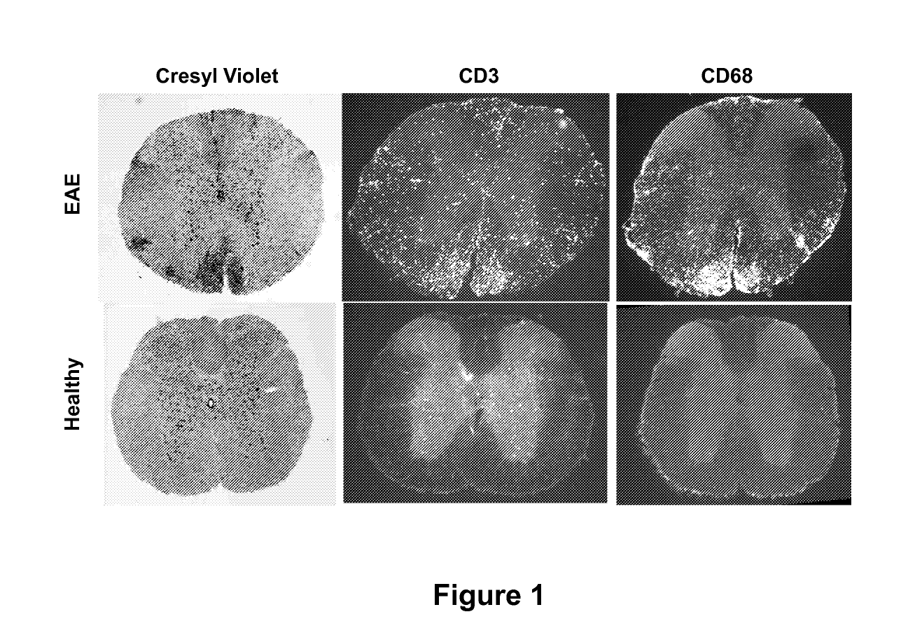

The observations leading to the invention were made using a well accepted animal model for MS, the experimental autoimmune encephalomyelitis (EAE) mouse. EAE is an experimental disease state that shares many clinical and pathological features with MS in humans. Many FDA-approved MS therapies were first discovered and developed based on the EAE models in mice and rats (reviewed by Steinman and Zamvil, 2006). Active immunization of EAE mice with several different protein components of myelin, including myelin basic protein (MBP), proteolipid protein (PLP), and myelin oligodendrocyte glycoprotein (MOG), induces the production of autoimmune antibodies and the clinical symptoms of ascending paralysis. For example, EAE can be induced in C57BL/6 mice following immunization with MOG peptide amino acids 35-55 (Aharoni, R., et al.) or PLP peptide amino acids 139-151 (PLPp (139-151)). Immunization with MOG or PLP induces myelin-specific autoimmune reactions, which cause demyelination and morbidity similar to that of MS. The clinical features of EAE include inflammation and demyelination of the CNS by large numbers of infiltrating lymphocytes, monocytes and macrophages (FIG. 1).

### Animal Experiments Using GLP-1R Agonists

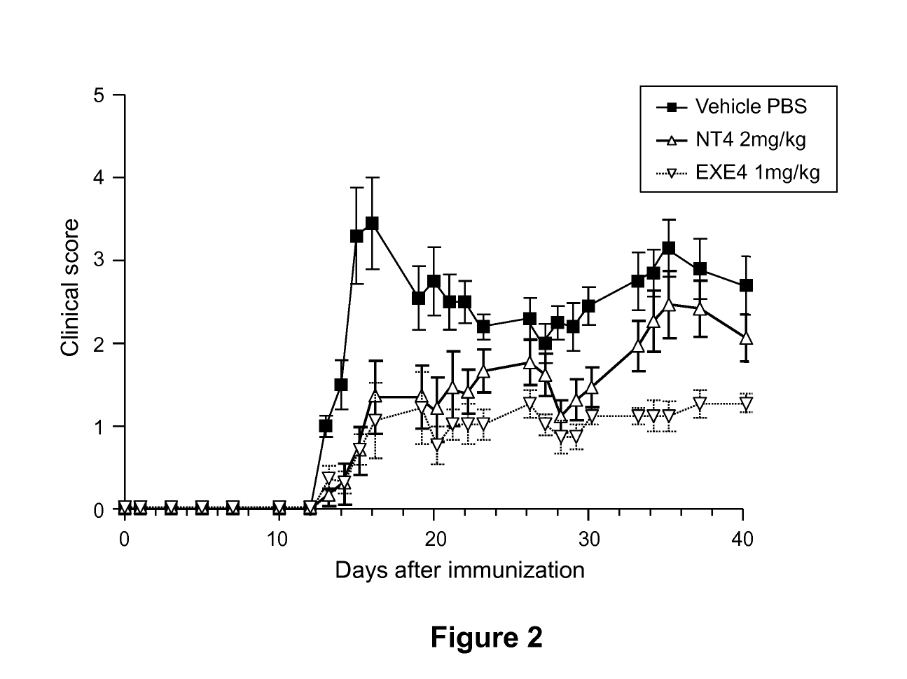

In experiments carried out in support of the present invention, direct administration of a GLP-1R agonist was shown to reduce morbidity and CNS tissue damage in animals with a CNS-specific autoimmune disease (FIGS. 2-4). Exendin-4 (EXE4) was administered to EAE animals following MOG immunization. The animals treated with exendin-4 showed significantly reduced morbidity compared to control animals. This result demonstrated that administration of a GLP-1R agonist was effective in slowing the progression of chronic EAE. Experiments indicated that the protection afforded by exendin-4 is at least as good as (if not better than) the protection afforded by trkB agonist NT-4 (see, PCT Application No. WO 2008/078179), with respect to the onset of symptoms, and that treatment with exendin-4 does not have to continue to the time of symptom onset in order for treatment to be effective (FIG. 2).

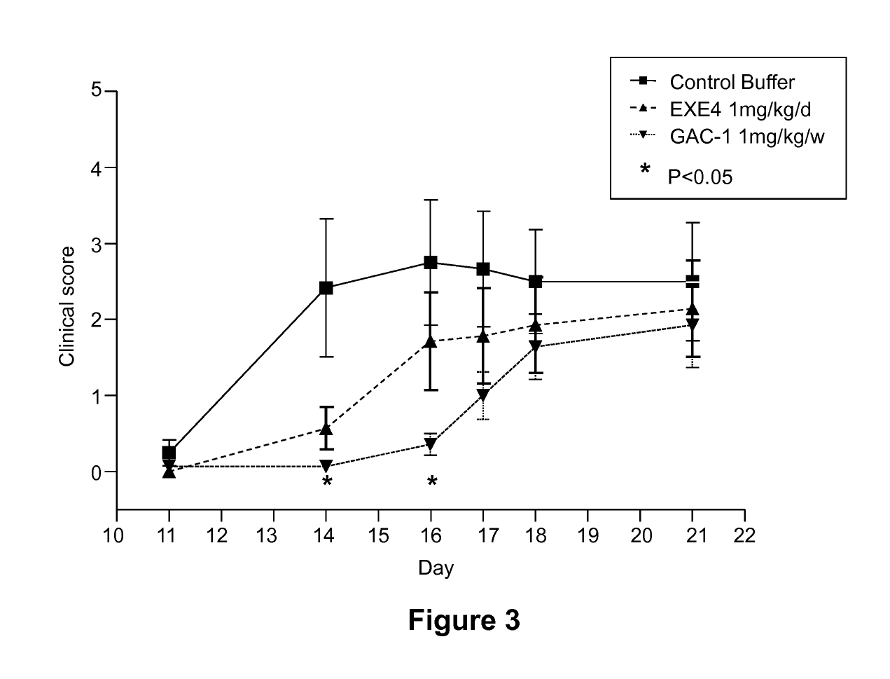

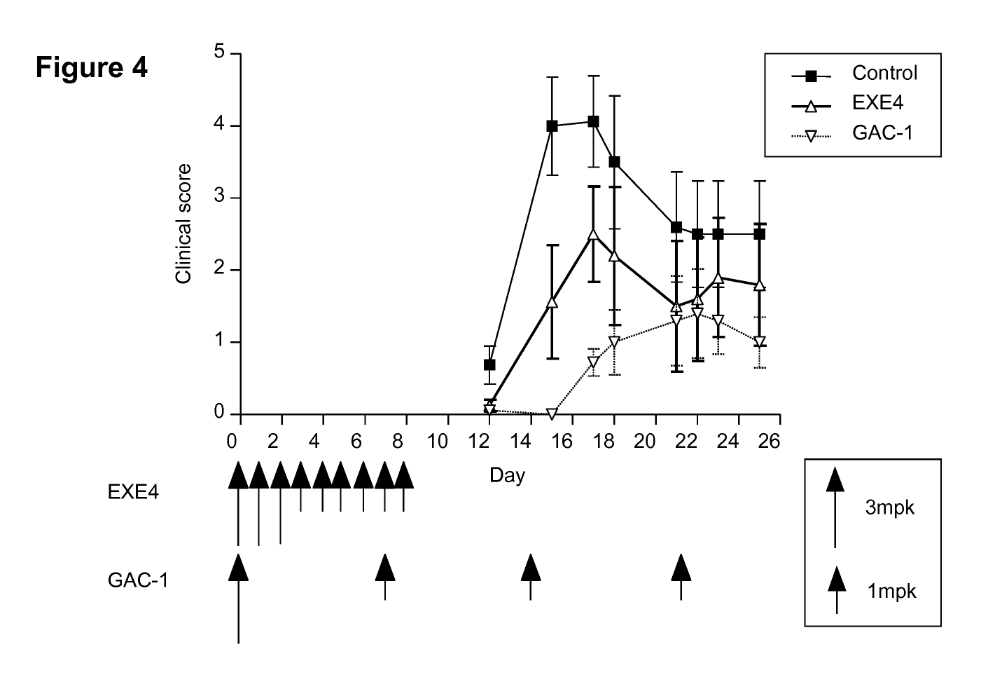

Animal experiments showed that weekly administration of a GLP-1R agonist compound, GAC-1, provided significant protection against EAE morbidity compared to a control (FIGS. 3 and 4). GAC-1 was effective when administered on a weekly basis. Experiments indicated that weekly treatment with GAC-1 protected at least as well as daily exendin-4 treatment up to the time of symptom onset.

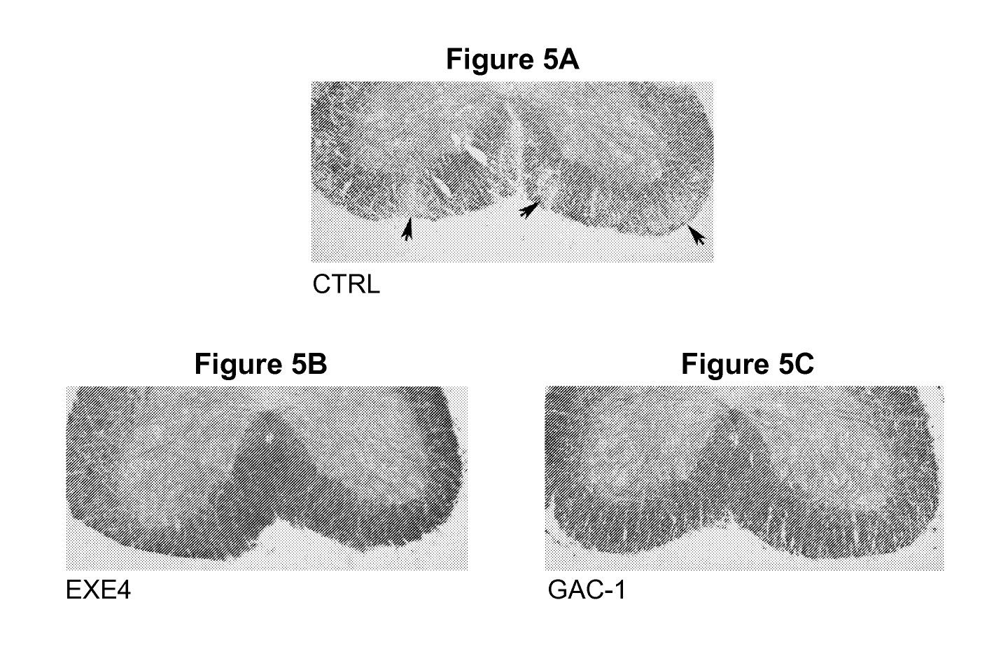

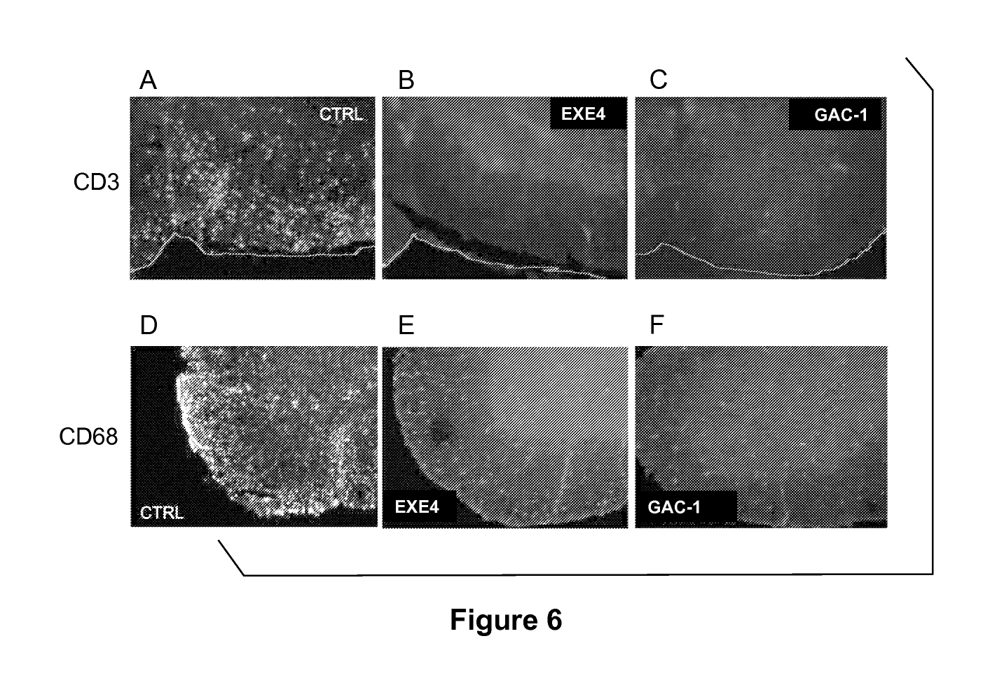

Histological analysis showed reduced demyelination and leukocyte invasion in sections prepared from animals treated with a GLP-1R agonist compared to sections from control EAE animals (FIGS. 5A-C and 6A-F). GLP-1R agonist-treated animals showed virtually no evidence of CNS CD3+ cell invasion. Identification of the invading cells was performed by staining for CD3-expressing T cells and CD68-expressing macrophages. Spinal cord tissues from GLP-1R agonist-treated animals show substantially reduced T cell and monocyte invasion when compared to control animals (FIGS. 6A-F). The results of the histochemical staining of CNS tissue sections demonstrated that GLP-1R agonist treatment reduces lymphocyte and monocyte invasion of the CNS.

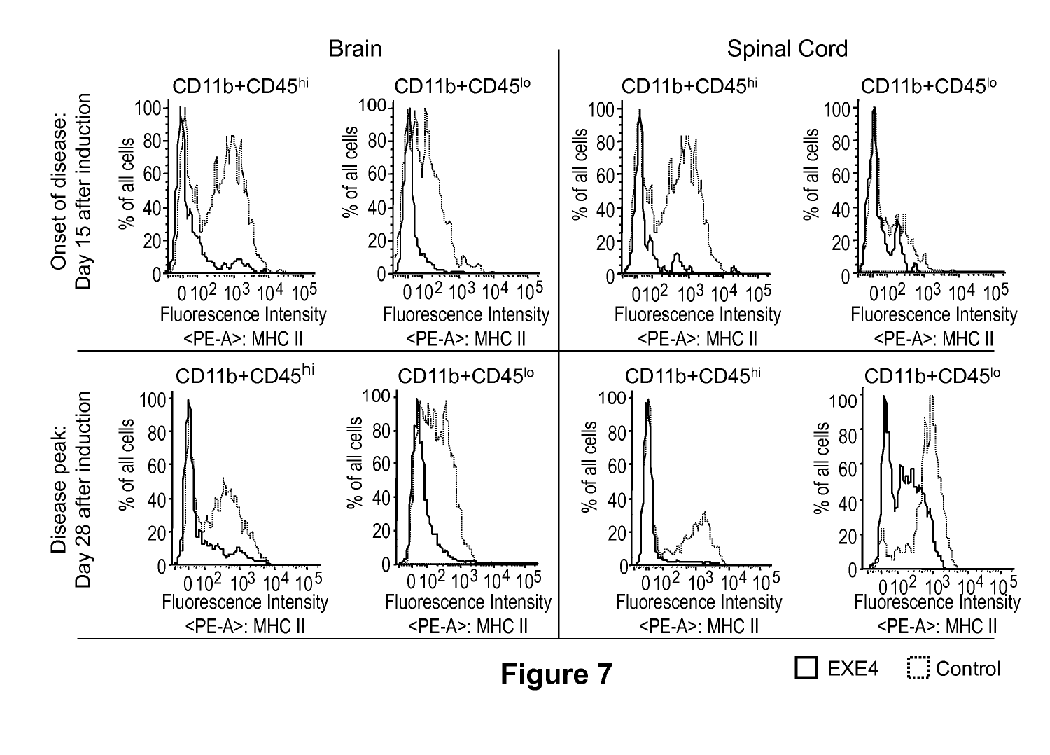

Analysis of activation marker MHC class II expression on microglia of EAE mice indicated treatment with exendin-4 reduced the expression level of MHC class II on both CD11b+CD45^{hi} (activated microglia and infiltrating macrophages) and CD11b+CD45^{lo} (resting microglia) cell populations in both brain and the spinal cord (FIG. 7). Resting microglia, activated microglia and infiltrating macrophages from EAE mice that received exendin-4 showed decreased MHC class II expression compared to cells from vehicle treated control animals at both symptom onset and disease peak.

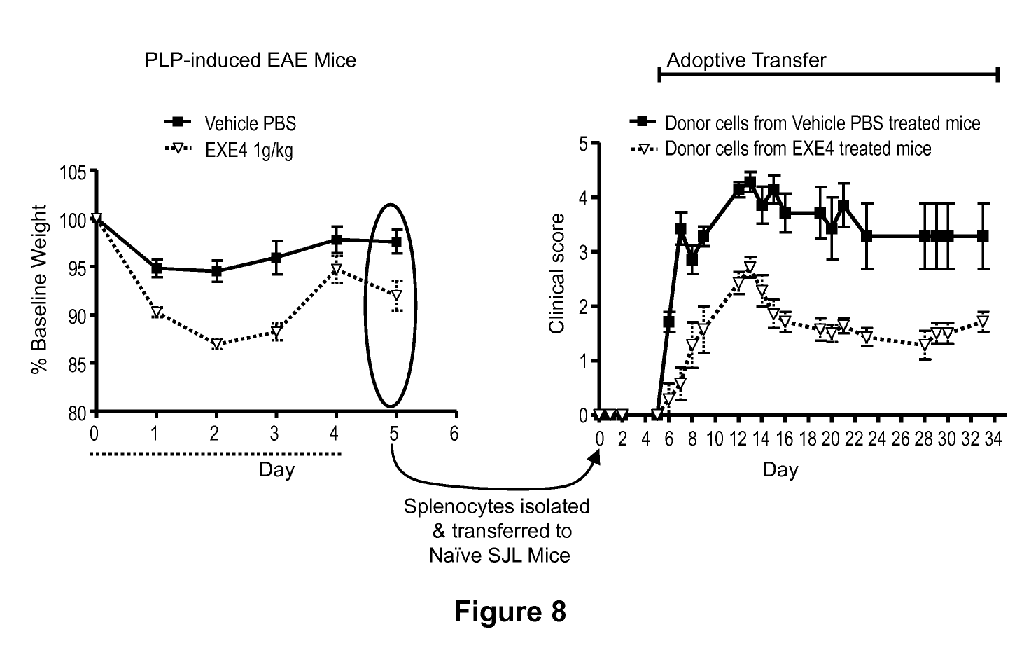

Animal experiments showed that splenocytes from exendin-4-treated mice are less pathogenic in an adoptive transfer model of EAE (FIG. 8). Mice that received splenocytes isolated from exendin-4 treated PLP-induced mice developed substantially less severe disease than mice that received splenocytes isolated from control (PBS-treated) PLP-induced mice.

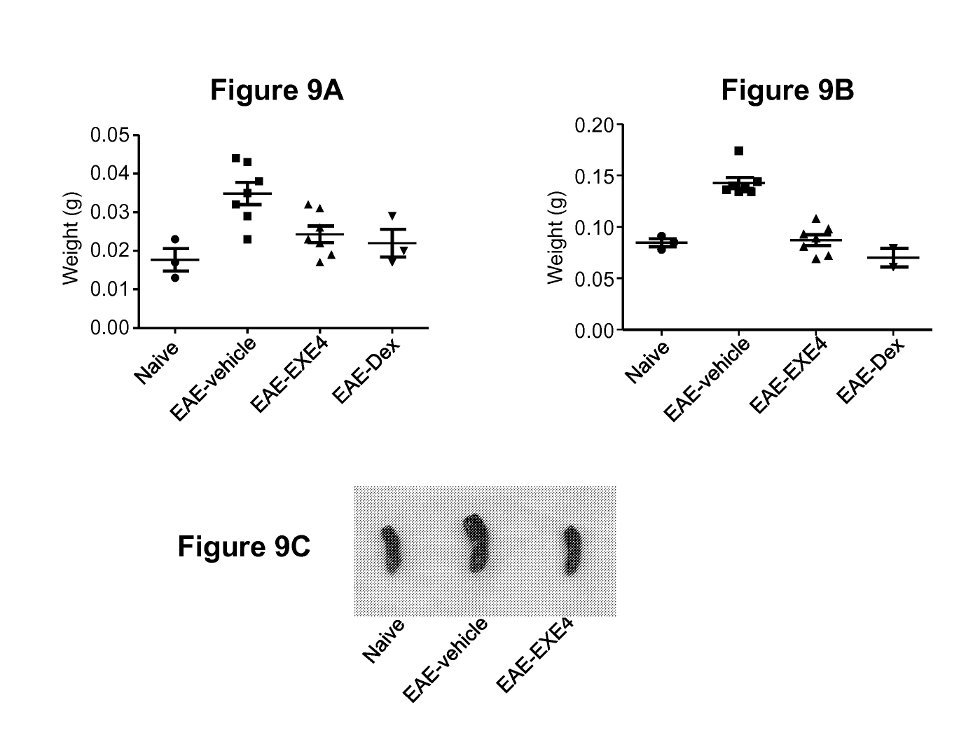

Inguinal lymph nodes of GLP-1R agonist-treated EAE mice weigh less than lymph nodes of vehicle treated control EAE animals (FIG. 9A). Lymph nodes of control treated EAE animals are substantially heavier. The spleen of GLP-1R agonist-treated EAE mice is comparable in weight and size to the spleen of naïve animals, in contrast to the spleen of vehicle treated control EAE animals, which is substantially heavier and larger (FIGS. 9B-C).

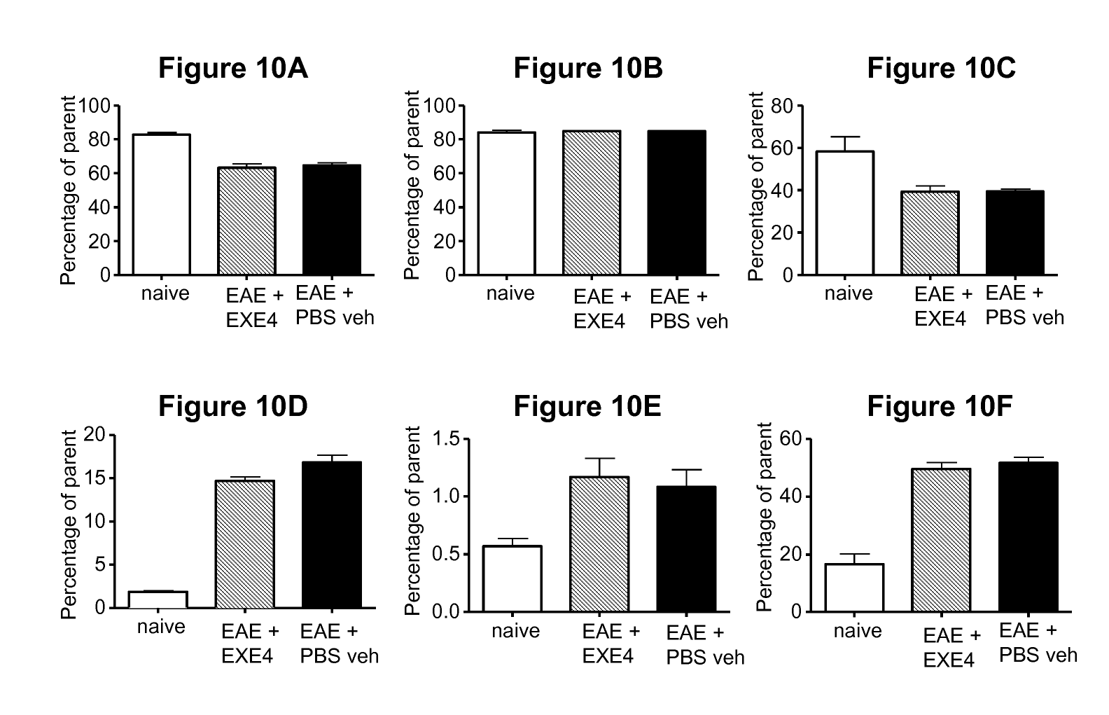

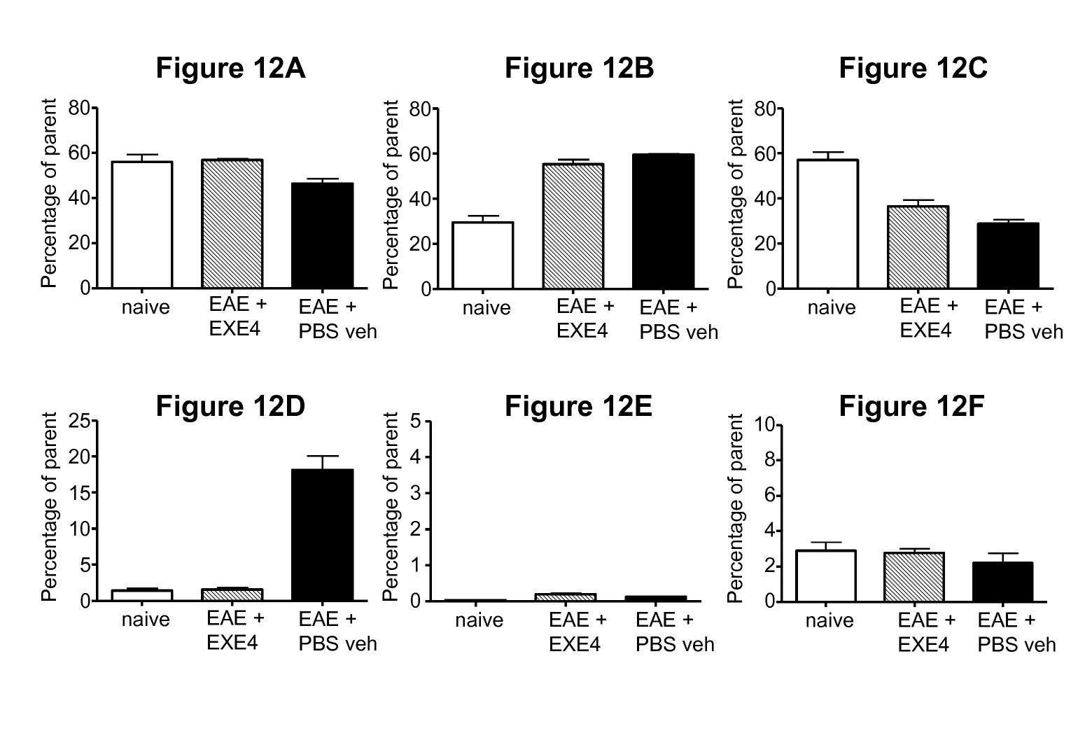

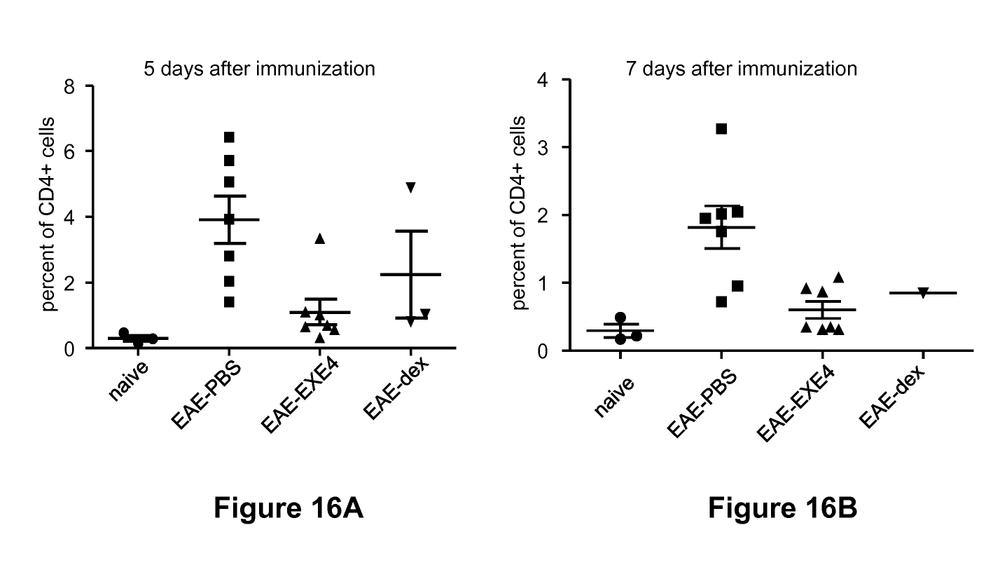

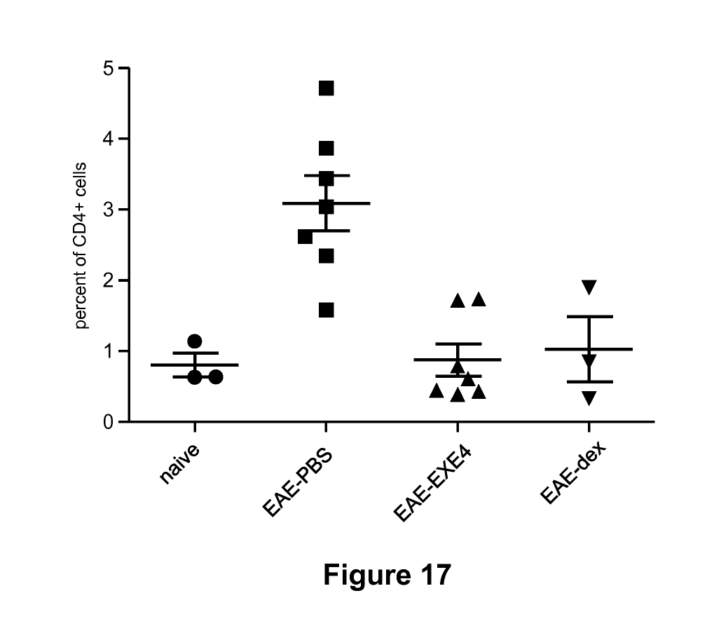

Animal experiments were carried out to analyze immunological changes resulting from GLP-1R agonist treatment of EAE animals (FIGS. 10A-F, 11A-F and 12A-F). GLP-1R agonist treatment resulted in a large reduction of Ter119+ cell numbers in MOG induced animals to close to Ter119+ cell numbers of naïve animals (FIGS. 12D-F and 13). In contrast, PBS treated control EAE animals have increased Ter119+ cell numbers. In addition, GLP-1R agonist treatment reduced IL-17+ and IFN-γ+ cell numbers in EAE animals in comparison to PBS treated control EAE animals, which have increased IL-17+ and IFN-γ+ cell numbers compared to naïve animals (FIGS. 16 and 17). The results of the immunological analysis suggest that GLP-1R agonist treatment reduces helper T cell levels in EAE animals.

The results described above demonstrate that GLP-1R agonists are effective in reducing demyelination and leukocyte invasion of the CNS and slowing the progression of EAE, a widely accepted animal model for MS. The naturally-occurring GLP-1R agonist exendin-4 and the GLP-1R agonist GAC-1 were both effective in slowing disease progression. Histochemical experiments showed that GLP-1R agonists reduce invasion of CNS tissues by T cells and monocytes.

### GLP-1R Agonists for CNS Autoimmune Disorders

Without being limited to a theory, it is believed that GLP-1R agonists function primarily by modulating the migration of leukocytes and/or reducing Th1 and Th17 cell levels. The present methods may be combined with current immunosuppressant treatments to produce additional therapeutic effects.

The GLP-1R agonists of the invention can be used to reduce leukocyte invasion of CNS tissues in a number of autoimmune or related diseases. The GLP-1R agonists of the invention can also be used to reduce Th1 and Th17 cell levels in a number of autoimmune or related diseases. In addition to being the model for MS, the EAE mouse is also used to study optic neuritis. GLP-1R agonists are expected to alleviate CNS immune invasion in all these diseases and other autoimmune disorders mediated by, inter alia, leukocyte invasion and/or Th1 and Th17 cells. Note that the terms disease and disorder are used without distinction.

A feature of the invention is the direct administration of a GLP-1R agonist to an animal suffering from an autoimmune disease. While the preferred embodiments of the invention are described in terms of polypeptides, the invention encompasses the administration of polynucleotides encoding such GLP-1R agonist polypeptides as will direct the expression of the encoded-GLP-1R agonists in the body. Methods of direct DNA injection and gene therapy delivery are known in the art. GLP-1R agonist polypeptides, or polynucleotides encoding them, are administered directly to an animal, as opposed to being induced by the administrations of a drug. The invention also encompasses peptidomimetic molecules that bind and activate GLP-1R in a manner consistent with naturally-occurring GLP-1R agonists and/or agonist antibodies.

Particular GLP-1R agonists for use according to the methods described herein are described in further detail below. Additional GLP-1R agonists will be apparent to one skilled in the art without departing from the scope of the invention.

### Naturally Occurring GLP-1R Agonists and Their Derivatives

GLP-1R agonists include naturally-occurring agonist polypeptides, fragments, variants, and derivatives, thereof, including, but not limited to GLP-1, exendin-4, proexendin (see, e.g., U.S. Pat. No. 6,723,530), exendin-3 (see, e.g., U.S. Pat. No. 5,424,286), OAP-189 (see, e.g., U.S. Patent Appl. Publication No. US 20090181885, incorporated herein by reference in its entirety), and oxyntomodulin. As used herein, GLP-1 and/or exendin-4 polypeptide sequences may be from the same species as the corresponding GLP-1R or from a different species, provided that the resulting polypeptide binds to GLP-1R and functions as an agonist.

GLP-1R agonists include naturally-occurring and variant GLP-1. GLP-1 polypeptides have been identified in a number of mammals. HAEGTFTSDVSSYLEGQAAKEFIAWLVKGR (SEQ ID NO: 1) is the 30 amino acid GLP-1 (7-36) peptide generated by cleavage of GLP-1 by dipeptidyl peptidase IV (DPP-4) at the position 2 alanine. D. J. Drucker (2001) Endocrinology 142:521-527. GLP-1 (7-36) functions as a GLP-1R agonist, resulting in increased glucose-dependent insulin secretion. However, the half-life of GLP-1 (7-36) is only a few minutes. Protease cleavage sites may be removed to extend the half-life of GLP-1 polypeptides, or added to allow the regulation of their activity. GLP-1R agonists may be conjugated or fused to half-life extending moieties, such as a PEG, the IgG Fc region, albumin, or a peptide or epitopes such as Myc, HA (hemagglutinin), His-6, or FLAG. Other exemplary GLP-1 polypeptides include GLP-1(7-37) HAEGTFTSDVSSYLEGQAAKEFIAWLVKGR (SEQ ID NO: 42) and GLP-1(1-45) (see, e.g., U.S. Patent Application Publication No. 2003/0232754).

GLP-1R agonists further include naturally-occurring and variant exendin-4. Exendin-4 has been identified in the saliva of the Gila monster. Exendin-4 is a 39 amino acid peptide which is approximately 53% homologous to GLP-1. The sequence of Gila monster exendin-4 is HGEGTFTSDLSKQMEEEAVRLFIEWLKNGGPSSGAPPPS (SEQ ID NO: 2). Like GLP-1 (7-36), exendin-4 functions as a GLP-1R agonist and stimulates glucose-dependent insulin secretion. Unlike GLP-1 (7-36), however, exendin-4 shows increased resistance to cleavage by DPP-4. The N-terminal regions of GLP-1 (7-36) and exendin-4 are nearly identical, with the notable difference being the second amino acid residue. This residue is an alanine in GLP-1 (7-36), but a glycine in exendin-4. This single amino acid in the N-terminal region is responsible for the increased resistance of exendin-4 to DPP-4 digestion. Another notable difference between exendin-4 and DLP-1 (7-36) is the presence of nine additional amino acid residues at the C-terminus of exendin-4, which form a Trp-cage.

Naturally-occurring and variant GLP-1 and exendin-4 polypeptides of the invention include chimeras, variants, fragments (including peptides), and/or derivatives thereof. Preferred fragments include the GLP-1R-binding portion of a naturally-occurring polypeptides, or a chimeric, consensus, or mutated equivalent binding portion. Fragments include synthetic peptides. Variants include naturally-occurring amino acid sequence variants having conservative and non-conservative amino acid substitutions. Exemplary variants include, without limitation, GLP-1-Tf (U.S. Patent Application Pub. No. 20060205037), OAP-189, Exendin-4-Tf (WO2008012629) and GLP-1 and exendin-4 peptide analogues disclosed in U.S. Patent Application Publication No. 20040242853. The sequence of OAP-189 is HAQGTFTSDYSKYLEQELVKYFIQWLKNAGPSKNNIA (SEQ ID NO: 43).

Conservative substitutions involve amino acid residues of similar size, charge, or hydrophobicity. For example, Ala may be substituted by Val, Leu, or Ile. Arg may be substituted by Lys, Gln, or Asn. Asn may be substituted by Gln, His, Lys, or Arg. Asp may be substituted by Glu. Cys may be substituted by Ser. Gln may be substituted by Asn. Glu may be substituted by Asp. Gly may be substituted by Pro. His may be substituted by Asn, Gln, Lys, or Arg. Ile may be substituted by Leu, Val, Met, Ala, Phe, or Norleucine. Leu may be substituted by Norleucine, Ile, Val, Met, Ala, or Phe. Lys may be substituted by Arg, Gln, or Asn. Met may be substituted by Leu, Phe, or Ile. Phe may be substituted by Leu, Val, Ile, or Ala. Pro may be substituted by Gly. Ser may be substituted by Thr. Thr may be substituted by Ser. Trp may be substituted by Tyr. Tyr may be substituted by Trp, Phe, Thr, or Ser. Val may be substituted by Ile, Leu, Met, Phe, Ala, or Norleucine.

Substantial modifications in function may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties (some of these may fall into several functional groups):
(1) Hydrophobic: Met, Ala, Val, Leu, Ile, Norleucine;
(2) Neutral hydrophilic: Cys, Ser, Thr;
(3) Acidic: Asp, Glu;
(4) Basic: Asn, Gln, His, Lys, Arg;
(5) Aromatic: Trp, Tyr, Phe; and
(6) Residue that induce bending: Gly and Pro.

Non-conservative substitutions exchange a member of one class for a member of another class, or involve a substitution not identified as conservative in the previous paragraphs.

Any cysteine residue not involved in maintaining the proper conformation of the agonist also may be substituted, generally with serine, to improve the oxidative stability of the molecule and prevent aberrant cross-linking. Conversely, cysteine bond(s) may be added to the agonist to improve its stability.

Amino acid modifications can range from changing or modifying one or more amino acids to complete redesign of a region, such as the variable region. Changes in the variable region can alter binding affinity and/or specificity.

Variants include naturally-occurring amino acid sequence variants and engineered variants, provided that the resulting polypeptides or derivatives bind to GLP-1R and function as agonists. Assays for measuring GLP-1R activation are described herein and in the references cited.

Derivatives include covalently and non-covalently modified peptides and polypeptides (e.g., acylated, pegylated, farnesylated, glycosylated, or phosphorylated) polypeptides. The polypeptides may include additional functional groups to modulate binding and/or activity, allow imaging in the body, modulate half-life, modulate transport across the blood-brain barrier, or assist in the targeting of the polypeptide to a particular cell type or tissue. The polypeptides may comprise amino acid substitutions to facilitate modification (e.g. the addition of pegylation, glycosylation, or other sites), provided that the substitutions do not substantially affect the binding of the polypeptide to GLP-1R or agonist activity.

A common modification is pegylation to reduce systemic clearance with minimal loss of biological activity. Polyethylene glycol polymers (PEG) may be linked to various functional groups of the GLP-1 and exendin-4 polypeptides (as well as GLP-1R agonist antibodies) using methods known in the art (see, e.g., Roberts et al. (2002), Advanced Drug Delivery Reviews 54:459-476; Sakane et al. (1997) Pharm. Res. 14:1085-91). PEG may be linked to, e.g., amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 kDa). PEG molecules linked to GLP-1, exendin-4, or other polypeptides may have a molecular weight about any of 2000, 10,000, 15,000, 20,000, 25,000, 30,000, 35,000, 40,000 Da. PEG molecule may be a single or branched chain. To link PEG to GLP-1R agonist polypeptides, a derivative of the PEG having a functional group at one or both termini may be used. The functional group is chosen based on the type of available reactive group on the polypeptide. Methods of linking derivatives to polypeptides are known in the art. One example of an N-terminal specific attachment for PEG is to mutate the residue at position 1 to a serine or threonine to facilitate pegylation.

Polypeptides or their derivatives may be linked to other molecules directly or via synthetic linkers. Preferred GLP-1R agonist polypeptides, fragments, or derivatives, thereof, exhibit similar or better binding affinity, selectivity, and activation compared to naturally-occurring GLP-1R agonists for which values have been reported. Small portions of the agonist polypeptides may be referred to as “peptides,” although this terminology should not be construed as limiting.

Preferred GLP-1R agonists exhibit similar biological properties compared to exendin-4 and GAC-1 in the numerous experiments and assays described herein, including the EAE animal model.

### GLP-1R Agonist-Antibody Conjugates

Derivatives further include covalent linkage of a GLP-1R agonist peptide to an antibody to extend half-life and/or enhance potency. For example, GLP-1R agonist compounds comprising a GLP-1R agonist peptide agent linked, either directly or via an intervening linker, to a combining site of an antibody, are described in, e.g., U.S. Patent Application Pub. No. 20090098130 and PCT Application Pub. No. WO 2008/081418, which are both incorporated herein by reference in their entireties. Such GLP-1R agonist compounds are referred to herein as “GLP-1R agonist-Antibody Conjugates” or “GACs.” A GAC includes a GLP-1R agonist peptide, such as, e.g., a GLP-1 or exendin-4 fragment or derivative, linked to an antibody either directly or via a linker. In some embodiments where a linker is used, the linker may act to distance the GLP-1R agonist peptide from the antibody to avoid the antibody limiting binding between the GLP-1R agonist peptide and GLP-1R.

GACs as disclosed herein may include any GLP-1R agonist peptide, i.e., a peptide that increases the amount of activation of GLP-1R, producing effects similar to those produced by the naturally-occurring agonists GLP-1 and exendin-4. In some embodiments, a GLP-1R peptide may be flanked by one or more R groups. For example, in some embodiments, the GLP-1R agonist peptide of a GAC may have the structure: R¹—HAEGTFTSDVSSYLEGQAAKEFIAWLVKGR (SEQ ID NO: 1)-R², wherein: R¹ is absent, CH₃, C(O)CH₃, C(O)CH₂CH₃, C(O)CH₂CH₂CH₃, or C(O)CH(CH₃)CH₃; and R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate. In other embodiments, the GLP-1R agonist peptide may have the structure: R¹—HGEGTFTSDLSKQMEEEAVRLFIEWLKNGGPSSGAPPPS (SEQ ID NO: 2)-R² (SEQ ID NO: 4); wherein R¹ is absent, CH₃, C(O)CH3, C(O)CH₂CH₃, C(O)CH₂CH₂CH₃, or C(O)CH(CH₃)CH₃; and R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate. GLP-1R agonist peptides further include analogs of SEQ ID NOs: 1 and 2. Such analogs may possess additional advantageous features, such as, for example, increased bioavailability, increased stability, improved EAE treatment profile, improved Th17 cell number reduction profile, improved appetite control, improved body weight control, and/or reduced host immune recognition. As used herein, an analog of a peptide of SEQ ID NO: 1 or SEQ ID NO: 2 is a peptide having essentially the sequence of SEQ ID NO: 1 or SEQ ID NO: 2, but with one or more amino acid substitutions, insertions, or deletions, or a combination thereof.

In certain embodiments, GLP-1R agonist peptides as provided herein comprise SEQ ID NO: 1 or SEQ ID NO: 2. Suitable exemplary SEQ ID NO: 1 and SEQ ID NO: 2 analogs are set forth in, e.g., Table II of U.S. patent application Ser. No. 11/969,850, filed Jan. 4, 2008 (published as U.S. Pat. Appl. Pub. No. 20090098130), which is incorporated herein by reference in its entirety.

A GLP-1R agonist peptide can be prepared using techniques well known in the art. For example, methods of manufacturing GLP-1R agonist antibody conjugate compounds are described in U.S. Pat. Appl. Pub. No. 20090098130. Typically, synthesis of the GLP-1R agonist peptide is the first step, and is carried out as described in U.S. Pat. Appl. Pub. No. 20090098130. The GLP-1R agonist peptide is then derivatized for linkage to a connecting component (the linker), which is then combined with the antibody. One of skill in the art will readily appreciate that the specific synthetic steps used depend upon the exact nature of the three components. Thus, the GLP-1R agonist peptid-linker conjugates and GACs described herein can be readily synthesized.

“Antibody” as used herein includes immunoglobulins which are the product of B cells and variants thereof as well as the T cell receptor (TcR) which is the product of T cells and variants thereof. The combining site refers to the part of an antibody molecule that participates in antigen binding.

A GLP-1R agonist peptide as herein described may be covalently linked to a combining site in an antibody either directly or via a linker. An appropriate linker can be chosen to provide sufficient distance between the targeting agent and the antibody. Other linker considerations include the effect on physical or pharmacokinetic properties of the resulting GAC or GLP-1R agonist peptide-linker, solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like. It has been found that even minor alterations to peptide composition, linker composition and linking residue location can have unpredictable and surprising effects on different characteristics of the resultant molecule (e.g., stability, solubility, half-life, bioavailability, target binding ability, agonist or antagonist activity, pharmokinetics, etc.).

In some embodiments, peptides for use with the present invention may be of Formula I: R¹—[H¹X²E³G⁴T⁵F⁶T⁷S⁸D⁹X¹⁰S¹¹X¹²X¹³X¹⁴E¹⁵X¹⁶X¹⁷A¹⁸X¹⁹X²⁰X²¹F²²X²³X²⁴X²⁵X²⁶X²⁷X²⁸X²⁹X³⁰X³¹X³²X³³X³⁴X³⁵X³⁶X³⁷X³⁸X³⁹X⁴⁰(SEQ ID NO: 3)]-R², wherein: X² is a blocking group such as Aib, A, S, T, V, L, I, D-Ala; X¹⁰ is V, L, I, or A; X¹² is S or K; X¹³ is Q or Y; X¹⁴ is G, C, F, Y, W, M, or L; X¹⁶ is K, D, E, or G; X¹⁷ is E or Q; X¹⁹ is L, I, V, or A; X²⁰ is ornithine or a derivatized lysine group such as K(SH)R, or K; X²¹ is L or E; X²³ is I or L; X²⁴ is A or E; X²⁵ is W or F; X²⁶ is L or I; X²⁷ is I, K, or V; X²⁸ is R, ornithine, N, or K; X²⁹ is Aib or G; X³⁰ is any amino acid, preferably G or R; X³¹ is P or absent; X³² is S or absent; X³³ is S or absent; X³⁴ is G or absent; X³⁵ is A or absent; X³⁶ is P or absent; X³⁷ is P or absent; X³⁸ is P or absent; X³⁹ is S or absent; and X⁴⁰ is a linking residue or absent; and wherein one of X¹⁰, S¹¹, X¹², X¹³, X¹⁴, X¹⁶, X¹⁷, X¹⁹, X²⁰, X²¹, X²⁴, X²⁶, X²⁷, X²⁸, X³², X³³, X³⁴, X³⁵, X³⁶, X³⁷, X³⁸, X³⁹, or X⁴⁰ is substituted with a linking residue (-[LR]—) comprising a nucleophilic side chain covalently linkable to the combining site of an antibody directly or via an intermediate linker, and the linking residue may be selected from the group consisting of K, R, Y, C, T, S, homologs of lysine (including K(SH)), homocysteine, and homoserine.

In some embodiments, peptides for use with the invention comprise the sequence of Formula II: R¹—[H¹-(Aib)²-E³G⁴-T⁵-F⁶-T⁷-S⁸-D⁹-V¹⁰-S¹¹-S¹²Y¹³-L¹⁴-E¹⁵-G¹⁶-Q¹⁷-A¹⁸-A¹⁹-K²⁰-E²¹-F²²-I²³-A²⁴-W²⁵-L²⁶-V²⁷-K²⁸-G²⁹-R³⁰ (S EQ ID NO: 4)]-R² In some embodiments, one of the residues may be substituted with the linking residue. In some embodiments, one of G¹⁶ or A²⁴ may be substituted with the linking residue, comprising a nucleophilic side chain covalently linkable to the combining site of an antibody directly or via an intermediate linker, and the linking residue may be selected from the group consisting of K, R, Y, C, T, S, homologs of lysine (including K(SH)), homocysteine, and homoserine, In other embodiments, the linker may be covalently attached to the C-terminus of the R³⁰ group, or the C-terminus of an additional residue at position 31.

In some embodiments, peptides for use with the invention may comprise the sequence of Formula III: R¹-[H¹-(Aib)²-E³-G⁴-F⁶-T⁷-S⁸-D⁹-L¹⁰-S¹¹-K¹²-Q¹³-M¹⁴-E¹⁵-E¹⁶-E¹⁷-A¹⁸-V¹⁹-R²⁰-L²¹-F²²-I²³-E²⁴-W²⁵_{-L}²⁶-K²⁷-N²⁸-G²⁹-G³⁰-P³¹-S³²-S³³-G³⁴-A³⁵-P³⁶-P³⁷-P³⁸-S³⁹-X⁴⁰ (SEQ ID NO: 5)]-R², wherein X⁴⁰ is a linking residue or absent, and wherein one of L¹⁰, S¹¹, K¹², Q¹³, M¹⁴, E¹⁶, E¹⁷, A¹⁹, R²⁰, L²¹E²⁴, L²⁶, K²⁷, N²⁸, G³², S³³, G³⁴, A³⁵, P³⁶, P³⁷, P³⁸, S³⁹, or X⁴⁰ is substituted with a linking residue (-[LR]—) comprising a nucleophilic side chain covalently linkable to the combining site of an antibody directly or via an intermediate linker, wherein the linking residue may be selected from the group consisting of K, R, Y, C, T, S, homologs of lysine (including K(SH)), homocysteine, and homoserine.

For Formulae I, II and III, X² may be a blocking residue, such as Aib, or may be alanine, (for example as found in GLP1), or glycine, (for example as found in exendin-4), or another residue.

For Formulae I, II and III, R¹ may be absent, CH₃, C(O)CH3, C(O)CH₂CH₃, C(O)CH₂CH₂CH₃, or C(O)CH(CH₃)CH₃; and R² may be absent, OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

For Formulae I, II and III; R¹ may be absent. For Formulae I, II and III; R² may be NH₂.

For Formulae I, II and III, the linking residue may be lysine (K). The linking residue may be C. For Formulae I, II and III, the linking residue may be K(SH):

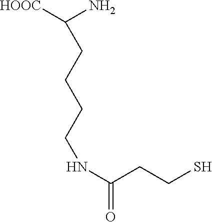

Where the linking residue is K, K(SH), or C, the linking residue may further be covalently attached to the following linker:

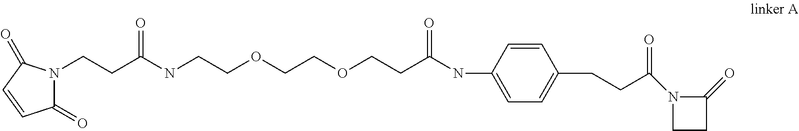

The linker may be covalently linked through the β-lactam group to an antibody as follows:

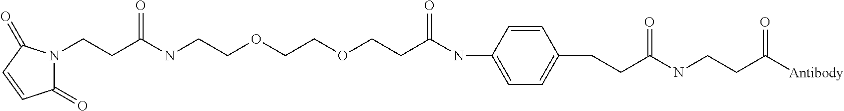

In some embodiments, the β-lactam group of the linker may be covalently linked to at least one of the two combining sites of an antibody as follows:

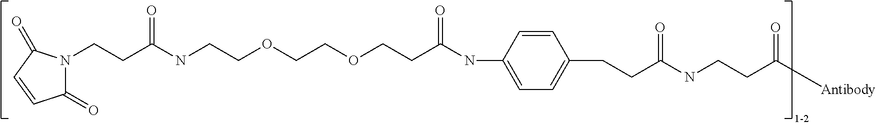

In some embodiments, the linking residue and linker may be of the structure:

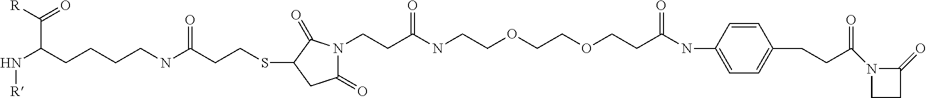

In some embodiments, the linker and linking residue may be covalently attached to a GLP-1R agonist peptide, such as those exemplified by Formulae I, II and III, as follows:

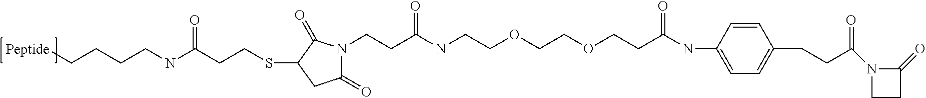

In some embodiments, the peptide-linker complex may be covalently attached to an antibody:

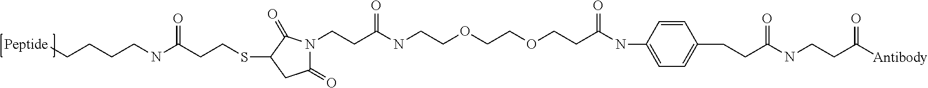

In some embodiments, the peptide-linker complex is attached to at least one of the combining sites of an antibody:

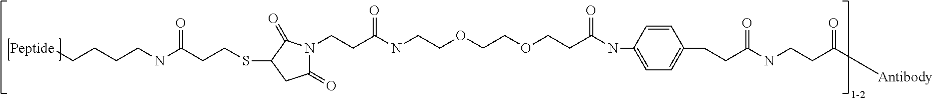

In certain embodiments, the GLP-1R agonist peptide may comprise the sequence: H¹-(Aib)²-E³-G⁴-T⁵-F⁶-T⁷-S⁸-D⁹-L¹⁰-S¹¹-K¹²-Q¹³-M¹⁴-E¹⁵-E¹⁶-E¹⁷-A¹⁸-V¹⁹-R²⁰-L²¹-F²²-I²³-E²⁴-W²⁵-L²⁶-K²⁷-N²⁸-G²⁹-G³⁰-P³¹-S³²-S³³-G³⁴-A³⁵-P³⁶-P³⁷-P³⁸-S³⁹-X⁴⁰ (SEQ ID NO: 6), wherein one of M¹⁴ or X⁴⁰ is substituted with the linking reside. In some embodiments, the GLP-1R agonist peptide-linker comprises the peptide NH₂-[H-(Aib)-E-G-T-F-T-S-D-L-S-K-Q-M-E-E-E-A-V-R-L-F-I-E-W-L-K-N-G-G-P-S-S-G-A-P-P-P-S-K (SEQ ID NO: 7)]-NH₂ linked to linker A via K⁴⁰ (the linking residue) as shown below:

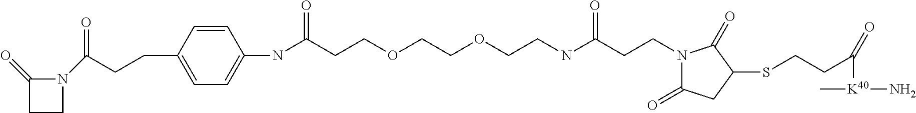

In compound GAC-1, the peptide NH₂-[H-(Aib)-E-G-T-F-T-S-D-L-S-K-Q-M-E-E-E-A-V-R-L-F-I-E-W-L-K-N-G-G-P-S-S-G-A-P-P-P-S-K (SEQ ID NO: 7)1-NH₂ is linked to linker A via K⁴⁰ (the linking residue) and bound to the combining site of an antibody as shown below:

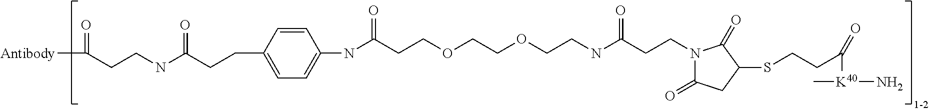

Accordingly, GAC-1 has the following structure:

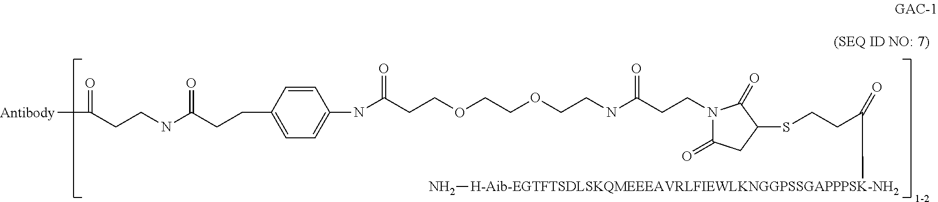

In other embodiments, the GLP-1R agonist peptide-linker comprises the peptide NH₂-[H-(Aib)-E-G-T-F-T-S-D-L-S-K-Q-K-E-E-E-A-V-R-L-F-I-E-W-L-K-N-G-G-P-S-S-G-A-P-P-P-S (SEQ ID NO: 8)]-NH₂ linked to linker A via K¹⁴ (the linking residue) as shown below:

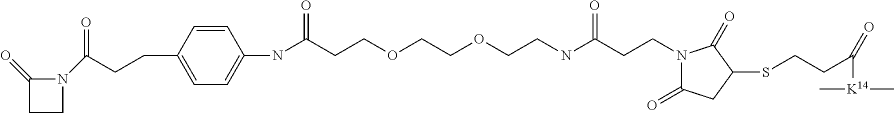

In compound GAC-2, the peptide NH₂-[H-(Aib)-E-G-T-F-T-S-D-L-S-K-Q-K-E-E-E-A-V-R-L-F-I-E-W-L-K-N-G-G-P-S-S-G-A-P-P-P-S (SEQ ID NO: 8)]-NH₂ is linked to linker A via K¹⁴ (the linking residue) and bound to the combining site of an antibody as shown below:

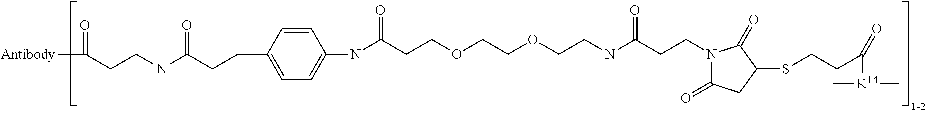

Accordingly, GAC-2 has the following structure:

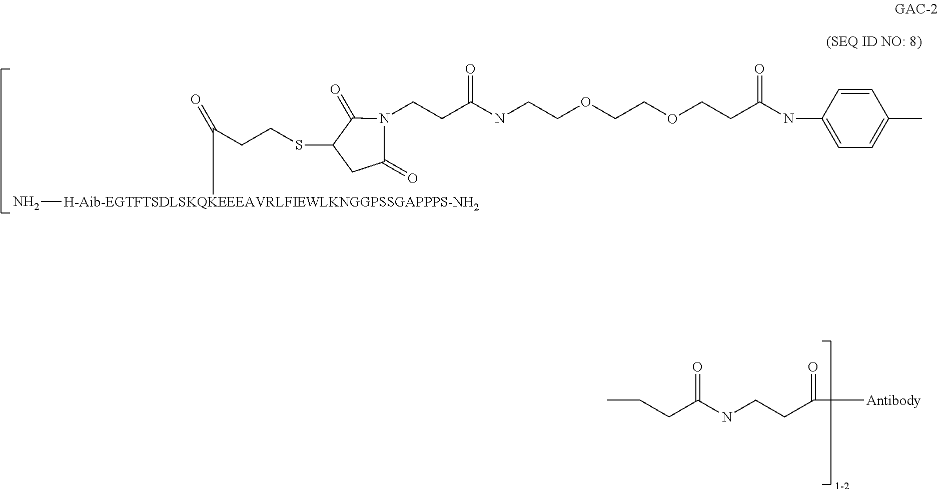

Exemplary GACs include, without limitation, a GAC having any of the peptide sequences provided in Table 1 below. In some embodiments, the GLP-1R agonist compound is any GAC from Table 1. Data presented in Table 1 was obtained using methods described in U.S. Pat. Appl. Pub. No. 20090098130. The EC₅₀ values provided in Table 1 were determined using a glucose-stimulated insulin secretion (GSIS) assay as described in, e.g., Example 30 of U.S. Pat. Appl. Pub. No. 20090098130. In Table 1, (P) in the EC₅₀ column indicates EC₅₀ for when the peptide is tethered to an antibody.

TABLE 1

| | | Linking | EC₅₀ | T_{1/2} (IV) | % bio- |
|---|---|---|---|---|---|
| GAC- | Sequence | residue | (μM) | Mouse | available |
| | HAEGTFTSDVSSYLEGQAAKEFIA | N/A | 0.051 | ND | ND |
| | WLVKGR (SEQ ID NO: 1) | (peptide | | (not | |
| | | only) | | done) | |
| | HGEGTFTSDLSKQMEEEAVRLFIE | N/A | 0.029 | ND | ND |
| | WLKNGGPSSGAPPPS (SEQ ID | (peptide | | | |
| | NO: 2) | only) | | | |
| 1 | H(Aib)EGTFTSDLSKQMEEEAVRL | C-term | 0.078, | 20.7 | 85 |
| | FIEWLKNGGPSSGAPPPS [symbol] (SEQ | | 2.65 (P) | | |
| | ID NO: 7) | | | | |
| 2 | H(Aib)EGTFTSDLSKQ [symbol] EEEAVRL | K14 | 0.033 | 65.8 | ~100 |
| | FIEWLKNGGPSSGAPPPS (SEQ | | 2.45 (P) | | |
| | ID NO: 8) | | | | |
| 3 | H(Aib)EGTFTSDVSSYLEGQAAKE | C-term | 0.04, | 12.8 | ND |
| | FIAWLVKGR [symbol] (SEQ ID NO: 9) | | 2.66 (P) | | |
| 4 | H(Aib)EGTFTSDVSSYLEGQAA [symbol] E | K26 | 0.041, | 24.5 | 27 |
| | FIAWLVKGR (SEQ ID NO: 10) | | 1.04 (P) | | |
| 5 | H(Aib)EGTFTSDVSSYLEGQAAKE | C-term | ND | 11.2 | ND |
| | FIAWLVKGR [symbol] (SEQ ID NO: 13) | | | | |
| 6 | H(Aib)EGTFTSDVSSYLEGQAA [symbol] E | K26 | ND | 21.3 | ND |
| | FIAWLVK(Aib)R (SEQ ID NO: 14) | | | | |
| 7 | H(Aib)EGTFTSDVSSYLEGQAAKE | C-term | ND | 21.0 | ND |
| | FIAWLVK(Aib)R [symbol] (SEQ ID NO: 15) | | | | |
| 8 | H(Aib)EGTFTSDVSSYLEGQAA [symbol] E | K26 | ND | 21.2 | ND |
| | FIAWLVK(Aib)R (SEQ ID NO: 16) | | | | |
| 9 | H(Aib)EGTFTSDVSSYLEGQAA [symbol] E | K26 | 0.094, | ND | ND |
| | FIAWLVK(Aib)R (SEQ ID NO: 17) | | 3.35 (P) | | |
| 10 | H(Aib)EGTFTSD [symbol] SSYLEEQAVKE | K16 | ND | 36.9 | ND |
| | FIAWLIK(Aib)R (SEQ ID NO: 18) | | | | |
| 11 | H(Aib)EGTFTSDVSSYLEEQ [symbol] VKE | K24 | ND | 35.2 | ND |
| | FIAWLIK(Aib)R (SEQ ID NO: 19) | | | | |
| 12 | H(Aib)EGTFTSDVSSYLEEQAVKE | C-term | ND | 25.3 | 32 |
| | FIAWLIK(Aib)RPSSGAPPPS [symbol] | | | | |
| | (SEQ ID NO: 20) | | | | |
| 13 | H(Aib)EGTFTSDVSSYLEEQAV [symbol] E | K26 | ND | 40.7 | ND |
| | FIAWLIK(Aib)RPSSGAPPPS (SEQ | | | | |
| | ID NO: 21) | | | | |
| 14 | HGEGTFTSDLSKQMEEEAVRLFIE | C-term | 0.026 | ND | ND |
| | WLKNGGPSSGAPPPS [symbol] (SEQ ID | | | | |
| | NO: 22) | | | | |
| 15 | H(Aib)EGTFTSDLSKQMEEEAVRL | K38 | ND | 29.2 | 39 |
| | FIEWLKNGGPSSGAPP [symbol] S (SEQ | | | | |
| | ID NO: 23) | | | | |
| 16 | H(Aib)EGTFTSDLSKQMEEEAVRL | K36 | ND | ND | ND |
| | FIEWLKNGGPSSGA [symbol] PPS (SEQ | | | | |
| | ID NO: 24) | | | | |
| 17 | H(Aib)EGTFTSDLSKQMEEEAVRL | K34 | ND | ND | ND |
| | FIEWLKNGGPSS [symbol] APPPS (SEQ | | | | |
| | ID NO: 25) | | | | |
| 18 | H(Aib)EGTFTSDLSKQMEEEAVRL | K32 | ND | ND | ND |
| | FIEWLKNGGP [symbol] SGAPPPS (SEQ | | | | |
| | ID NO: 26) | | | | |
| 19 | H(Aib)EGTFTSDLSKQMEEEAVRL | K28 | ND | 40.7 | 46 |
| | FIEWLK [symbol] GGPSSGAPPPS (SEQ | | | | |
| | ID NO: 27) | | | | |
| 20 | H(Aib)EGTFTSDLSKQMEEEAVRL | K27 | ND | 35.2 | 72 |
| | FIEWL [symbol] NGGPSSGAPPPS (SEQ | | | | |
| | ID NO: 28) | | | | |
| 21 | H(Aib)EGTFTSDLSKQMEEEAVRL | K26 | 0.01 | 65.5 | ~100 |
| | FIEW [symbol] KNGGPSSGAPPPS (SEQ | | | | |
| | ID NO: 29) | | | | |
| 22 | H(Aib)EGTFTSDLSKQMEEEAVRL | K24 | 0.14, no | 33.1 | 57 |
| | FI [symbol] WLKNGGPSSGAPPPS (SEQ | | fit | | |
| | ID NO: 30) | | | | |
| 23 | H(Aib)EGTFTSDLSKQMEEEAVRL | K23 | 0.03 | 114.3 | 82 |
| | F [symbol] EWLKNGGPSSGAPPPS (SEQ | | | | |
| | ID NO: 31) | | | | |
| 24 | H(Aib)EGTFTSDLSKQMEEEAVR [symbol] | K21 | 0.010, | 66.2 | 72 |
| | FIEWLKNGGPSSGAPPPS (SEQ | | 2.12(P) | | |
| | ID NO: 32) | | | | |
| 25 | H(Aib)EGTFTSDLSKQMEEEAV [symbol] L | K20 | 0.047 | 50.9 | 78 |
| | FIEWLKNGGPSSGAPPPS (SEQ | | | | |
| | ID NO: 33) | | | | |
| 26 | H(Aib)EGTFTSDLSKQMEEEA [symbol] RL | K19 | no fit, | 82.5 | ~100 |
| | FIEWLKNGGPSSGAPPPS (SEQ | | 7.60(P) | | |
| | ID NO: 34) | | | | |
| 27 | H(Aib)EGTFTSDLSKQMEE [symbol] AVRL | K17 | no fit | 58.7 | 48 |
| | FIEWLKNGGPSSGAPPPS (SEQ | | | | |
| | ID NO: 35) | | | | |
| 28 | H(Aib)EGTFTSDLSKQME [symbol] EAVRL | K16 | 0.01 | 54.1 | 88 |
| | FIEWLKNGGPSSGAPPPS (SEQ | | | | |
| | ID NO: 36) | | | | |
| 29 | H(Aib)EGTFTSDLSK [symbol] MEEEAVRL | K13 | 0.031, | 53.5 | 80 |
| | FIEWLKNGGPSSGAPPPS (SEQ | | 2.67(P) | | |
| | ID NO: 37) | | | | |
| 30 | H(Aib)EGTFTSDLS [symbol] QMEEEAVRL | K12 | 0.091, | 30.8 | 94 |
| | FIEWLKNGGPSSGAPPPS (SEQ | | 1.76 (P) | | |
| | ID NO: 38) | | | | |
| 31 | H(Aib)EGTFTSDL [symbol] KQMEEEAVRL | K11 | 0.08 | 52 | ~100 |
| | FIEWLKNGGPSSGAPPPS (SEQ | | | | |
| | ID NO: 39) | | | | |
| 32 | H(Aib)EGTFTSDLSKQMEEEAVRL | C-term | ND | 43.9 | 77 |
| | FIEWLKNGG [symbol] (SEQ ID NO: 40) | | | | |
| 33 | H(Aib)EGTFTSDLSKQMEEEAVR [symbol] | K21 | ND | 46.2 | 98 |
| | FIEWLKNGGPS (SEQ ID NO: 41) | | | | |

As discussed above, a compound for use with the invention may be covalently combined to an antibody. U.S. Patent Appl. Pub. No. 20090098130, incorporated herein by reference in its entirety, describes, inter alia, antibodies, useful fragments and variants and modifications thereof, combining sites and CDRs, antibody preparation, expression, humanization, amino acid modification, glycosylation, ADCC, CDC, increasing serum half life of antibodies, expression vectors, mammalian host systems, and folding, and other elements of antibody technology.

In some embodiments, some antibodies that can be used in conjunction with compounds of the invention may have a reactive side chain in the antibody combining site. A reactive side chain may be present naturally or may be placed in an antibody by mutation. The reactive residue of the antibody combining site may be associated with the antibody, such as when the residue is encoded by nucleic acid present in the lymphoid cell first identified to make the antibody. Alternatively, the amino acid residue may arise by purposely mutating the DNA so as to encode the particular residue (see, e.g., WO 01/22922 to Meares et al.). The reactive residue may be a non-natural residue arising, for example, by biosynthetic incorporation using a unique codon, tRNA, and aminoacyl-tRNA as discussed herein. In another approach, the amino acid residue or its reactive functional groups (e.g., a nucleophilic amino group or sulfhydryl group) may be attached to an amino acid residue in the antibody combining site. Thus, covalent linkage with the antibody occurring “through an amino acid residue in a combining site of an antibody” as used herein means that linkage can be directly to an amino acid residue of an antibody combining site or through a chemical moiety that is linked to a side chain of an amino acid residue of an antibody combining site. In some embodiments, the amino acid is cysteine, and the reactive group of the side chain is a sulfhydryl group. In other embodiments, the amino acid residue is lysine, and the reactive group of the side chain is the ε-amino group

Catalytic antibodies are one source of antibodies with suitable combining sites that comprise one or more reactive amino acid side chains. Such antibodies include aldolase antibodies, beta lactamase antibodies, esterase antibodies, amidase antibodies, and the like.

One embodiment comprises an aldolase antibody such as the mouse monoclonal antibodies mAb 33F12 and mAb 38C2, as well as suitably chimeric and humanized versions of such antibodies (e.g., h38C2, SEQ ID NOs: 11 and 12).

h38C2 light Chain sequence (219 amino acids):
(SEQ ID NO: 11)
ELQMTQSPSSLSASVGDRVTITCRSSQSLLHTYGSPYLNWYLQKPGQSP
KLLIYKVSNRFSGVPSRFSGSGSGTDFTLTISSLQPEDFAVYFCSQGTHL
PYTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
h38C2h chain sequence (448 amino acids):
(SEQ ID NO: 12)
EVQLVESGGGLVQPGGSLRLSCAASGFTFSNYWMSWVRQSPEKGLEW
VSEIRLRSDNYATHYAESVKGRFTISRDNSKNTLYLQMNSLRAEDTGIYY
CKTYFYSFSYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYT
QKSLSLSPGK

Mouse mAb 38C2 (and h38C2) has a reactive lysine near to but outside HCDR3, and is the prototype of a new class of catalytic antibodies that were generated by reactive immunization and mechanistically mimic natural aldolase enzymes. See C.F. Barbas 3^{rd} et al., Science 278:2085-2092, 1997). Other aldolase catalytic antibodies that may be used include the antibodies produced by the hybridoma 85A2, having ATCC accession number PTA-1015; hybridoma 85C7, having ATCC accession number PTA-1014; hybridoma 92F9, having ATCC accession number PTA-1017; hybridoma 93F3, having ATCC accession number PTA-823; hybridoma 84G3, having ATCC accession number PTA-824; hybridoma 84G11, having ATCC accession number PTA-1018; hybridoma 84H9, having ATCC accession number PTA-1019; hybridoma 85H6, having ATCC accession number PTA-825; hybridoma 90G8, having ATCC accession number PTA-1016. Through a reactive lysine, these antibodies catalyze aldol and retro-aldol reactions using the enamine mechanism of natural aldolases. See, e.g., J. Wagner et al., Science 270:1797-1800, 1995; C. F. Barbas 3^{rd} et al., Science 278:2085-2092, 1997; G. Zhong et al., Angew. Chem. Int. Ed. Engl. 38:3738-3741, 1999; A. Karlstrom et al., Proc. Natl. Acad. Sci. U.S.A., 97:3878-3883, 2000. Aldolase antibodies and methods of generating aldolase antibodies are disclosed in U.S. Pat. Nos. 6,210,938, 6,368,839, 6,326,176, 6,589,766, 5,985,626, and 5,733,75, which are incorporated herein by reference.

Compounds of the invention may also be formed by linking a compound of the invention to a reactive cysteine, such as those found in the combining sites of thioesterase and esterase catalytic antibodies. Suitable thioesterase catalytic antibodies are described by K. D. Janda et al., Proc. Natl. Acad. Sci. U.S.A. 91:2532-2536 (1994). Suitable esterase antibodies are described by P. Wirsching et al., Science 270:1775-1782 (1995). Reactive amino acid-containing antibodies may be prepared by means well known in the art, including mutating an antibody combining site residue to encode for the reactive amino acid or chemically derivatizing an amino acid side chain in an antibody combining site with a linker that contains the reactive group.

In some embodiments, the antibody may be a humanized antibody. Preferably, the humanized antibodies retain high linking affinity for the β-lactam group or other chemical group capable of forming a covalent linkage in the combining site. Various forms of humanized murine aldolase antibodies are contemplated. In some embodiments, the antibody is a humanized aldolase catalytic antibody such as h38c2 IgG1 or h38c2 Fab with human constant domains C_{K} and C_{γ1}1. C. Rader et al., 2003, J. Mol. Bio. 332:889-899 discloses the gene sequences and vectors that may be used to produce h38c2 Fab and h38c2 IgG1. Human germline Vₖ gene DPK-9 and human Jₖ gene JK4 were used as frameworks for the humanization of the kappa light chain variable domain of m38c2, and human germline gene DP-47 and human J_{H} gene JH4 were used as frameworks for the humanization of the heavy chain variable domain of m38c2. In certain embodiments of compounds of the invention comprising antibody h38c2 IgG1 with the G1m(f) allotype, the β-lactam group of the linker may bind to the side chain of the lysine residue at position 99 of the heavy chain. Other embodiments may use a chimeric antibody comprising the variable domains (V_{L} and V_{H}) of h38c2 and the constant domains from an IgG1, IgG2, IgG3, or IgG4. An antibody may be a full-length antibody, Fab, Fab′, F(ab′)₂, Fᵥ, dsFᵥ, scFᵥ, V_{H}, V_{L}, diabody, or minibody comprising V_{H} and V_{L} domains from h38c2. An antibody may also be an antibody comprising the V_{H} and V_{L} domains from h38c2 and a constant domain selected from the group consisting of IgG1, IgG2, IgG3, and IgG4. In some embodiments, an antibody may be h38C2 IgG1. In some embodiments, an antibody may be a humanized version of a murine aldolase antibody comprising a constant region from a human IgG, IgA, IgM, IgD, or IgE antibody. In other embodiments, the antibody can be a chimeric antibody comprising the variable region from a murine aldolase antibody and a constant region from a human IgG, IgA, IgM, IgD, or IgE antibody. In further embodiments, the antibody can be a fully human version of a murine aldolase antibody comprising a polypeptide sequence from natural or native human IgG, IgA, IgM, IgD, or IgE antibody.

Various forms of humanized aldolase antibody fragments are also contemplated. One embodiment uses h38c2 F(ab′)₂. h38c2 F(ab′)₂ may be produced by the proteolytic digestion of h38c2 IgG1. Another embodiment uses an h38c2 scFv comprising the V_{L} and V_{H} domains from h38c2 which are optionally connected by the intervening linker (Gly₄Ser)₃. As an alternative to humanization, human antibodies can be generated.

### Other GLP-1R Agonists

GLP-1R agonists also include, for example without limitation, anti-GLP-1R agonist antibodies, exenatide (BYETTA®), albiglutide, R1583, liraglutide, AVE-0010, S4P and Boc5 (see, Chen et al. (2007) Proc Natl Acad Sci USA. 104:943-948).

GLP-1R agonists also include DPP-4 inhibitors such as, for example without limitation, sitagliptin, vildagliptin, saxagliptin, linagliptin, dutogliptin, gemigliptin, alogliptin and berberine.

In some embodiments, the GLP-1R agonist is a monoclonal anti-GLP-1R agonist antibody. Monoclonal anti-GLP-1R antibodies can be produced readily by one skilled in the art. The general methodology for making monoclonal antibodies by hybridomas is now well known to the art. See, e.g., M. Schreier et al., Hybridoma Techniques (Cold Spring Harbor Laboratory 1980); Hammerling et al., Monoclonal Antibodies and T-Cell Hybridomas (Elsevier Biomedical Press 1981); Kennett et al., Monoclonal Antibodies (Plenum Press 1980). Immortal, antibody-secreting cell lines can also be produced by techniques other than fusion, such as direct transformation of B-lymphocytes with oncogenic DNA or EBV. Several antigen sources can be used, if desired, to challenge the normal B-lymphocyte population that is later converted to an immortal cell line.

### Polynucleotides

In some embodiments, the invention provides polynucleotides encoding any of the polypeptides described herein. Polynucleotides can be made and expressed by procedures known in the art.

In some embodiments, the invention provides compositions (such as a pharmaceutical compositions) comprising any of the polynucleotides of the invention. In some embodiments, the composition comprises an expression vector comprising a polynucleotide encoding the polypeptide as described herein. In other embodiment, the composition comprises an expression vector comprising a polynucleotide encoding any of the polypeptides described herein. Expression vectors and administration of polynucleotide compositions are further described herein.

In some embodiments, the invention provides a method of making any of the polynucleotides described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native polypeptide (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1× SSC containing EDTA at 55° C. The skilled artisan will recognize how to adjust the temperature, ionic strength, etc., as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989, supra.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston (1994).

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s), as disclosed, e.g., in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are useful, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to GLP-1R or a GLP-1R domain is effected by an immunoassay or FACS. A cell overexpressing the protein of interest can be identified.

### GLP-1R Agonist Compositions

GLP-1R agonists can be used in the manufacture of a medicament for the treatment of an autoimmune disease affecting the central nervous system, such as multiple sclerosis. In this manner, compositions comprising GLP-1R agonists may be used to treat a disease in a mammal (including a human patient), as defined herein.

The compositions used in the methods of the invention comprise an effective amount of a GLP-1R agonist. It is understood that the compositions can comprise more than one GLP-1R agonists.

GLP-1R agonist compositions may further comprise suitable pharmaceutically acceptable excipients. Suitable carriers, diluents and excipients are well known in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). Some formulations may include carriers such as liposomes. Liposomal preparations include, but are not limited to, cytofectins, multilamellar vesicles and unilamellar vesicles. Excipients and formulations for parenteral and nonparenteral drug delivery are set forth in Remington, The Science and Practice of Pharmacy (2000).

Generally, GLP-1R agonist compositions are formulated for administration by injection (e.g. intraperitoneal, intravenous, subcutaneous, intramuscular, etc.), although other forms of administration can be used. GLP-1R agonists can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### Administration and Dosing

The administration schedules and therapeutic dosages exemplified herein were based on such factors as animal body mass, the half-life of GLP-1R agonist in the blood, and the affinity of the GLP-1R agonists. Preferred administration schedules and dosages maintain a therapeutic amount of GLP-1R agonists in the body between administrations. Effective dosage ranges for exendin-4 and GAC-1 are exemplified herein, in Davies et al., 1993, and in other references.

As demonstrated by experiments performed in support of the invention, a “pulse treatment” with exendin-4 early in the course of the disease appears to be sufficient to reduce morbidity in animals. Continuing administration of the GLP-1R agonist may not be required for the efficacy of the treatment. In other embodiments, such as treatment with GACs, weekly or less frequent treatments may be sufficient for the efficacy of the treatment.

The particular dosage regimen, i.e., dose, timing and repetition, will depend on the age, condition, and body weight of the human or animal to be treated. Initial dosage regimens may be extrapolated from animal experiments. The timing/frequency of GLP-1R agonist administrations should be based on the circulating half-life (or the half-life in neuronal tissue) of a particular GLP-1R agonist, the amount of agonist that passes the blood-brain barrier, the half-life of the agonist in cells, toxicity, and side-effects, as they apply.

In the present studies, administration of GLP-1R agonists was performed by intraperitoneal (i.p.) injection; however, other routes of administration are expected to be effective (e.g., intravenous, subcutaneous, intramuscular). Intracranial or intraspinal/intrathecal administration (or administration to other tissues of the CNS) is likely to be effective. Other routes of administration may be suitable, depending on the particular GLP-1R agonist, its coating, conjugation, or particular biological properties (e.g., oral, sublingual, intrasynovial, mucosal, transdermal, intra-articular, vaginal, anal, intraurethral, nasal, aural, via inhalation, insufflation, via catheter, as a bolus, on a stent or other implantable device, in an embolic composition, in an intravenous drip, on a patch or dissolving film, etc.).

The GLP-1R agonist is preferably administered via a suitable peripheral route. Nonetheless, it is understood that a small percentage of the agonist may traverse the blood-brain barrier and be delivered to cells of the central nervous system. In some cases, the amount of peripherally administered GLP-1R agonist that gains access to the CNS is small (even less than 1%).

A feature of the invention is the direct administration of a GLP-1R agonist to a mammalian subject suffering from an autoimmune disease. “Direct” means that GLP-1R agonist polypeptides, or polynucleotides capable of directing the expression of such polypeptides, are delivered to an animal by a standard route of inoculation. GLP-1R agonists may be delivered to animals in combination with other pharmacological agents, including immunosuppressants such as glucocorticoids, cytostatic agents (e.g., alkylating agents, antimetabolites, methotrexate, azathioprine and mercaptopurine), cytotoxic antibodies (e.g., T cell receptor and IL-2 receptor-specific antibodies), B cell depleting therapies (e.g., anti-CD20 antibody/RITUXAN®, anti-BLyS antibody), drugs affecting T cell migration (e.g., anti-integrin alpha 4/beta 1 antibody/TYSABRI®), drugs that act on immunophilins (e.g., cyclosporine, tacrolimus, sirolimus, rapamicin), interferons (e.g., IFN-β), glatiramer/COPAXONE®, opioids, TNF-binding proteins (e.g., circulating receptors), mycophenolate, and other biological agents used to suppress an animal's immune responses to foreign antibodies or therapeutic antigens.

In treating mammal subjects, including humans, suffering from an autoimmune disease, a therapeutically effective amount of a GLP-1R agonist or a pharmaceutically acceptable derivative is administered. For example, a GLP-1R agonist may be administered as a daily intravenous infusion from about 0.1 mg/kg body weight to about 15 mg/kg body weight. Accordingly, one embodiment provides a dose of about 0.5 mg/kg body weight. Another embodiment provides a dose of about 0.75 mg/kg body weight. Another embodiment provides a dose of about 1.0 mg/kg body weight. Another embodiment provides a dose of about 2.5 mg/kg body weight. Another embodiment provides a dose of about 5 mg/kg body weight. Another embodiment provides a dose of about 10.0 mg/kg body weight. Another embodiment provides a dose of about 15.0 mg/kg body weight. Doses of a GLP-1R agonist or a pharmaceutically acceptable derivative should be administered in intervals of from about once per day to 2 times per week, or alternatively, from about once every week to once per month. In one embodiment, a dose is administered to achieve peak plasma concentrations of a GLP-1R agonist according to the invention or a pharmaceutically acceptable derivative thereof from about .002 mg/ml to 30 mg/ml. This may be achieved by the sterile injection of a solution of the administered ingredients in an appropriate formulation (any suitable formulation solutions known to those skilled in the art of chemistry may be used). Desirable blood levels may be maintained by a continuous infusion of a GLP-1R agonist according to the invention as ascertained by plasma levels measured by a validated analytical methodology.

One method for administering a GAC to an individual comprises administering a GLP-1R agonist peptide—linker conjugate to the individual and allowing it to form a covalent compound with a combining site of an appropriate antibody in vivo. The antibody portion of a GAC that forms in vivo may be administered to the individual before, at the same time, or after administration of the GLP-1R agonist peptide-linker conjugate. A GLP-1R peptide may include a linker/reactive moiety, or the antibody combining site may be suitably modified to covalently link to the targeting agent. Alternatively, or in addition, an antibody may be present in the circulation of the individual following immunization with an appropriate immunogen. For example, catalytic antibodies may be generated by immunizing with a reactive intermediate of the substrate conjugated to a carrier protein. See R. A. Lerner and C. F. Barbas 3^{rd}, 1996, Acta Chem. Scand. 50:672-678. In particular, aldolase catalytic antibodies may be generated by administering with keyhole limpet hemocyanin linked to a diketone moiety as described by P. Wirsching et al., 1995, Science 270:1775-1782 (commenting on J. Wagner et al., 1995, Science 270:1797-1800).

An advantage of GACs over naturally-occurring GLP-1R agonists is that the GACs tend to have relatively long circulating half-lives compared to circulating protein-ligands. For example, while naturally-occurring agonists may require daily administration, GACs may only require weekly administration.

Treatment with GLP-1R agonists can be combined with conventional treatments for multiple sclerosis and related disorders. Conventional drugs for the treatment and management of multiple sclerosis include but are not limited to: ABC (i.e., Avonex-Betaseron/Betaferon-Copaxone) treatments (e.g., interferon beta la (AVONEX®, REBIF®), interferon beta lb (BETASERON®, BETAFERON®), and glatiramer acetate) (COPAXONE®; chemotherapeutic agents (e.g., mitoxantrone (NOVANTRONE®), azathioprine (IMURAN®), cyclophosphamide (CYTOXAN®, NEOSAR®), cyclosporine (SANDIMMUNE®), methotrexate, and cladribine (LEUSTATIN®); corticosteroids and adreno-corticotrophic hormone (ACTH) (e.g., methylprednisolone (DEPO-MEDROL®, SOLU-MEDROL®), prednisone (DELTASONE®), prednisolone (DELTA-CORTEF®), dexamethasone (MEDROL®, DECADRON®), adreno-corticotrophic hormone (ACTH®), and corticotrophin (ACTHAR®); pain mediation (dysaesthesia) (e.g., carbamazepine (TEGRETOL®, EPITOL®, ATRETOL®, CARBATROL®), gabapentin (NEURONTIN®), topiramate (TOPAMAX®), zonisamide (ZONEGRAN®), phenytoin (DILANTIN®), desipramine (NORPRAMIN®), amitriptyline (ELAVIL®), imipramine (TOFRANIL®, IMAVATE®, JANIMINE®), doxepin (SINEQUAN®, ADAPIN®, TRIADAPIN®, ZONALON®), protriptyline (VIVACTIL®), cannabis and synthetic cannabinoids (MARINOL®), pentoxifylline (TRENTAL®), ibuprofen (NEUROFEN®), aspirin, acetaminophen, and hydroxyzine (ATARAX®); and other treatments (e.g., natalizumab (ANTEGREN®), alemtuzumab (CAMPATH-1H®), 4-aminopyridine (FAMPRIDINE®), 3,4 diaminopyridine, eliprodil, IV immunoglobin (GAMMAGARD®, GAMMAR-IV®), GAMIMUNE N®, IVEEGAM®, PANGLOBULIN®, SANDOGLOBULIN®, VENOGLOBULIN®).

### Kits

The invention also provides kits of parts (kits) for practicing the methods of the invention. Kits of the invention include one or more containers comprising a GLP-1R agonist described herein and instructions for use in accordance with any of the methods of the invention described herein. Generally, these instructions comprise a description of how to administer the GLP-1R agonist. The kit may further comprise instructions for identifying animals in need of treatment and for monitoring or measuring the effectiveness of treatment. The instructions generally include information relating to dosage, dosage scheduling (frequency of administration), and route of administration. The instructions supplied in the kits may be written or machine/computer readable as in the form of a data file or spreadsheet. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits may also comprise an apparatus for administering GLP-1R agonists, including syringes, needles, catheters, inhalers, pumps, alcohol swaps, gauze, CNS biopsy apparatus, histological antibodies and stains, etc. The components of the kit are sterilized as needed. Kits may also provide additional pharmaceutical agents, including but not limited to immunosuppressants, such as glatiramer acetate (GA) and dexamethasone. Kits may include date stamps, tamper-proof packaging, and radio frequency identification (RFID) tags or other inventory control features.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example, the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a GLP-1R agonist. The container may further comprise a second pharmaceutically active agent.

Kits may optionally provide additional components such as buffers and interpretive information. Normally, the kit comprises a container and a label or package insert(s) on or associated with the container.

### EXAMPLE

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1

### Identification of GLP-1R Agonists

GLP-1R agonists may be identified using art-recognized methods, including one or more of the following methods. For example, an assay to detect an increase in the concentration of intracellular cAMP may be used. This assay is suitable for qualitative or quantitative measurement of cAMP by, for example, measuring the expression of a reporter gene under regulation of the cAMP response element, as well as for identification and characterization of potential agonist or antagonists of a selected GPCR. Compounds which can induce a reporter gene to express the reporter are identified as GLP-1R agonists. One such assay employs the human embryonal nephric cell strain which is stably transfected with the expression vector of GLP-1R gene and an expression vector of luciferase reporter gene under the regulation of the cAMP response element, to detect its response to the candidate compound (Thorens, 1992, Cell Biology 89: 8641-8645; Drucker et al., 1987, Proc. Natl. Acad. Sci. U.S.A. Vol.84: 3434-3438).

The first stage of the assay involves conformational change of a GPCR, in the present case a GLP-1R, wherein the receptor is present in the cell membrane of a eukaryotic cell containing a reporter gene under regulation of the cAMP response element. The receptor may be an endogenous receptor or nucleic acid encoding the receptor, or a receptor construct, may be transformed into the cell. Typically, a first solid phase (e.g., a well of a first assay plate) is coated with a substantially homogeneous population of such cells (usually a mammalian cell line) so that the cells adhere to the solid phase. Often, the cells are adherent and thereby adhere naturally to the first solid phase. An analyte, such as a candidate agonist, is then added to the wells having the adherent cells, such that the receptor (e.g. GLP-1R) is exposed to (or contacted with) the analyte. This assay enables identification of agonist ligands for the GPCR of interest (e.g. GLP-1R). Following exposure to the analyte, the adhering cells are incubated under suitable conditions for a predetermined period of time to allow expression of the reporter gene (e.g., about six hours).

The cells are then ready to be subjected to the detection phase of the assay. The expression of the reporter gene is measured, e.g., by detection of luciferase activity. Detection of luciferase activity can be carried out by any of a variety of suitable methods which are known in the art.

Following initial identification, the agonist activity of a candidate (e.g., an anti-GLP-1R monoclonal antibody) can be further confirmed and refined by determining the concentration of intracellular cAMP. Typically, a first solid phase (e.g., a well of a first assay plate) is coated with a substantially homogeneous population of such cells (usually a mammalian cell line such as, e.g., HEK 293 cells) so that the cells adhere to the solid phase. The candidate is inoculated into the assay plate containing the cells and incubated under suitable conditions for, e.g., about 1 hour. The concentration of intracellular cAMP can be detected by any of a variety of suitable methods which are known in the art. For example, the concentration of intracellular cAMP can be detected according to the specification of the cAMP-Screen Direct® system kit (Applied Biosystems). Other cAMP assay kits are commercially available, such as the HitHunter™ cAMP XS+Assay (DiscoveRX®), an in vitro-based competitive immunoassay. Briefly, free cAMP from cell lysates compete for antibody binding against labeled ED-cAMP conjugate, a small peptide fragment of β-galactosidase (β-gal). In the absence of free cAMP, ED-cAMP conjugates are captured by the antibody and are unavailable for complementation, resulting in low signal. In the presence of free cAMP, antibody sites are occupied, leaving ED-cAMP conjugate free to complement with EA, forming active β-gal EFC enzyme for substrate hydrolysis to produce a chemiluminescent signal. A positive signal is generated in direct proportion to the amount of free cAMP bound by the antibody.

The agonist activity of a candidate (e.g., an anti-GLP-1R monoclonal antibody) can be further confirmed and refined by bioassays, known to test the targeted biological activities. For example, the ability of a candidate to agonize GLP-1R can be tested in an assay to determine insulin secretion. The ability of a candidate to stimulate insulin secretion may be determined by providing a candidate to cultured animal cells, such as the RIN-38 rat insulinoma cell line, and monitoring the release of immunoreactive insulin (IRI) into the media. Alternatively, one can inject a candidate into an animal and monitor plasma levels of immunoreactive insulin (IRI).

The presence of IRI can be detected through the use of, e.g., a radioimmunoassay which can specifically detect insulin. Any radioimmunoassay capable of detecting the presence of IRI may be employed; one such assay is a modification of the method of Albano, J. D. M., et al., 1972, Acta Endocrinol., 70:487-509. In this modification, a phosphate/albumin buffer with a pH of 7.4 is employed. The incubation is prepared with the consecutive addition of 500 μl of phosphate buffer, 50 μl of perfusate sample or rat insulin standard in perfusate, 100 μl of anti-insulin antiserum (Wellcome Laboratories; 1:40,000 dilution), and 100 μl of ¹²⁵I insulin, giving a total volume of 750 μl in a 10×75 mm disposable glass tube. After incubation for 2-3 days at 4° C., free insulin is separated from antibody-bound insulin by charcoal separation. The assay sensitivity is 1-2 uU/mL. In order to measure the release of IRI into the cell culture medium of cells grown in tissue culture, one preferably incorporates radioactive label into proinsulin. Although any radioactive label capable of labeling a polypeptide can be used, it is preferable to use ³H leucine in order to obtain labeled proinsulin.

To determine whether a candidate GLP-1R agonist has insulinotropic properties may also be determined by pancreatic infusion. The in situ isolated perfused rat pancreas assay is a modification of the method of Penhos, J. C., et al., 1969, Diabetes, 18:733-738. Fasted male Charles River strain albino rats, weighing 350-600 g, are anesthetized with an intraperitoneal injection of Amytal Sodium (Eli Lilly and Co.: 160 ng/kg). Renal, adrenal, gastric, and lower colonic blood vessels are ligated. The entire intestine is resected except for about four cm of duodenum and the descending colon and rectum. Therefore, only a small part of the intestine is perfused, minimizing possible interference by enteric substances with glucagon-like immunoreactivity. The perfusate is a modified Krebs-Ringer bicarbonate buffer with 4% dextran T70 and 0.2% bovine serum albumin (fraction V, and is bubbled with 95% O₂ and 5% CO₂. A nonpulsatile flow, 4-channel roller bearing pump (Buchler polystatic, Buchler Instruments Division, Nuclear-Chicago Corp.) is used, and a switch from one perfusate source to another is accomplished by switching a 3-way stopcock. The manner in which perfusion is performed, monitored, and analyzed follow the method of Weir, G. C., et al., 1974, J. Olin. Inestigat. 54:1403-1412, which is incorporated herein by reference.

### Example 2

### Large Numbers of Lymphocytes and Macrophages Infiltrate the CNS of Mice with Autoimmune Disease

This example illustrates infiltration of the CNS with inflammatory cells.

EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Animals (EAE and naïve) were sacrificed 18 days after MOG immunization. Spinal cords were harvested, fixed with 4% paraformaldehyde and cryo-sectioned at 20 μM. Immunohistochemistry were performed with primary antibodies against CD3 (Chemicon) and CD68 (Serotec). Goat anti rat secondary antibodies conjugated to Alexa 488 (Invitrogen) were used for detection. Cresyl violet and luxol fast blue staining were used to stain infiltrating cell bodies and myelin.

The staining results in FIG. 1 show that the CNS of EAE mice (top panel) have large numbers of infiltrating T cells, monocytes and microglia compared to healthy mice (bottom panel).

### Example 3

### Direct Administration of a GLP-1R Agonist Reduces Morbidity in Autoimmune Disease

In experiments carried out in support of the present invention, direct administration of a GLP-1R agonist was shown to reduce morbidity and CNS tissue damage in animals with a CNS-specific autoimmune disease (FIG. 2). EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v.

Mice were treated daily from day 0 to day 9 after MOG immunization with 1 mg/kg exendin-4 (n=10), 2 mg/kg NT4 (positive control, n=10) or vehicle (PBS, negative control, n=10). Animals were scored for morbidity as follows: 0=normal; 1=limp tail; 2=moderate hind limb weakness; 3=moderately severe hind limb weakness (animal can still walk with difficulty); 4=severe hind limb weakness (animal can move its hind limbs but cannot walk); 5=complete hind limb paralysis; and 6=death. The animals treated with exendin-4 showed significantly reduced morbidity compared to control animals (FIG. 2). This result demonstrated that a GLP-1R agonist was effective in slowing the progression of chronic EAE.

### Example 4

### A GLP-1R Agonist Compound is Effective in Reducing Morbidity in Autoimmune Disease

Animal experiments showed that the GLP-1R agonist antibody conjugate compound GAC-1 provided significant protection against EAE morbidity compared to a control immunoglobulin (FIGS. 3 and 4).

EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Mice were treated daily from day 0 to day 6 after MOG immunization with either 1 mg/kg exendin-4 (n=10) or vehicle (n=10). Another group of mice were treated on a weekly basis with 1mg/kg GAC-1 (n=10). The mice were assessed daily for clinical signs of EAE according to the following scale: 0=normal; 1=limp tail; 2=moderate hind-limb weakness; 3=moderately severe hind-limb weakness (animal can still walk); 4=severe hind-limb weakness (animal can move their hind-limbs but cannot walk); 5=complete hind limb paralysis; 6=death. GAC-1 was effective in reducing morbidity when administered weekly following MOG immunization (FIG. 3).

In a separate experiment, EAE was induced as described above. A first group of animals were treated from day 0 to day 2 after MOG immunization with 3 mg/kg exendin-4 (n=10), and from day 3 to day 8 with 1mg/kg exendin-4. A second group of animals were treated with 3 mg/kg GAC-1 on day 0 after MOG immunization, followed by weekly treatments of 1 mg/kg GAC-1 starting on day 7 after immunization. A third group of animals were treated daily with PBS (control, n=10). Clinical scores were monitored daily following criteria described above. GAC-1 appeared to be even more effective in reducing morbidity when administered at an initial dose of 3 mg/kg followed by weekly doses of 1 mg/kg (FIG. 4). The results demonstrated that an initial dose of 3 mg/kg GAC-1 provides more protection from morbidity (i.e., less morbidity) than doses of 1 mg/kg alone.

### Example 5

### GLP-1R Aqonists Block Inflammatory Cell Invasion of the CNS and Reduce Demyelination in Autoimmune Disease

Histological analysis was performed to better understand the mechanism by which GLP-1R agonists mediate protection in animals. Spinal cord sections were prepared from control, exendin-4-treated, and GAC-1-treated EAE-induced animals, and then stained with Luxol fast blue to stain myelin, or antibodies specific for leukocyte markers to identify invading cells. In the control animals, the stainings showed a region of leukocyte invasion and cell destruction against a background of myelin-expressing neuronal cells (FIGS. 5A, 6A and 6D). Reduced leukocyte invasion was observed in sections prepared from animals treated with the GLP-1R agonists (FIGS. 6B, 6C, 6E and 6F). Some GLP-1R agonist-treated animals showed virtually no evidence of CNS leukocyte invasion.

The spinal cord sections were stained with Luxol Fast Blue to measure demyelination (FIGS. 5A-C). EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Animals were treated daily with 1 mg/kg exendin-4 or PBS control, or weekly with 1 mg/kg GAC-1. Animals were sacrificed 18 days after MOG immunization. Spinal cords were harvested, fixed with 4% paraformaldehyde and cryo-sectioned at 20 μM. Myelin staining was performed using Luxol Fast Blue.

FIGS. 5A-C depict representative images of spinal cords from PBS treated control EAE animals (FIG. 5A), exendin-4 treated EAE animals (FIG. 5B) and GAC-1 treated EAE animals (FIG. 5C). The brains of control mice showed regions of severe demyelination (i.e., the absence of Luxol Fast Blue staining, as indicated by the black arrows in FIG. 5A). Regions of severe demyelination and/or neuron death were not observed in sections from GLP-1R agonist-treated animals (FIGS. 5B and C). The results of the histochemical staining of CNS tissue sections demonstrated that GLP-1R agonist treatment reduces demyelination of the CNS.

Identification of the invading cells was performed using antibodies specific for two leukocyte markers, CD3 present on T cells and CD68 present on macrophages. EAE was induced in female C57BL/6 mice as described above. Animals were treated daily with 1 mg/kg exendin-4 or PBS control, or weekly with 1 mg/kg GAC-1. Animals were sacrificed 12 days after MOG immunization. Spinal cords were harvested, fixed with 4% paraformaldehyde and cryo-sectioned at 20 pM. Immunohistochemistry was performed with primary antibodies against CD3 (Chemicon) or CD68 (Serotech). Goat anti-rat secondary antibodies conjugated to Alexa 488 (Invitrogen) was used for detection.

FIGS. 6A-C show the results of staining with the CD3 antibody. Numerous brightly staining punctuate regions are apparent in spinal cord of control animals corresponding to areas of demyelination (FIG. 6A). In contrast, the spinal cord sections obtained from exendin-4 or GAC-1-treated mice showed virtually no staining for either CD3 (FIGS. 6B and 6C, respectively), indicating that T cell invasion was substantially reduced in GLP-1R agonist-treated animals. Such observations indicated that spinal cord tissues from GLP-1R agonist-treated animals show substantially reduced T cell invasion when compared to control animals.

FIGS. 6D-F show the results of staining with the CD68 antibody. The sections prepared from control mice (FIG. 6D) stained more strongly than the sections from exendin-4 or GAC-1-treated mice (FIGS. 6E and 6F, respectivley). Such observations indicated that spinal cord tissues from GLP-1R agonist-treated animals show substantially reduced monocyte invasion when compared to control animals. Taken together, the results of the immunohistochemical staining of CNS tissue sections demonstrated that GLP-1R agonist treatment reduces lymphocyte and monocyte invasion of the CNS.

### Example 6

### GLP-1R Agonists Reduce Activation Markers in Microglia and Infiltrating Macrophages in the Brain and Spinal Cord

This example illustrates GLP-1R agonist-mediated reduction of MHC-II expression, a marker of activation, in microglia and infiltrating macrophage in the brain and spinal cord both at the disease onset (day 15 after MOG induction) and at the peak of disease (day 28 after MOG induction).

To assess the effect of exendin-4 on antigen presenting cells (APC) in the CNS, mice were administered with MOG on day 0. MOG-induced animals received daily administrations of exendin-4 (1 mg/kg) or vehicle control from day 0 to day 9 (n=9 each treatment group). Three exendin-4 treated EAE mice and three vehicle-treated EAE control mice were sacrificed at day 15 (onset of disease) and day 28 (disease peak). Microglia were isolated from the sacrificed animals. Briefly, mice were anesthetized with isoflurane and perfused through the left ventricle with cold sterile PBS until the effluent ran clear. Spinal cord and brain were collected in cold Hank's buffered salt solution (HBSS). Spinal cord and brain were forced through a nylon cell strainer (100 um) with HBSS. The collected strained matter was pelleted by centrifugation. The pellet was resuspended in 1 ml of HBSS per spinal cord or brain with 300 U/ml per cord of Type IV clostridial collagenase. After incubation for 60 min in a 37° C. incubator, spinal cord or brain homogenates were placed in 30% Percoll and underlaid with 70% Percoll. The gradients were centrifuged, and mononuclear cells were collected from the 30%/70% Percoll interface. The cells were washed from the interface twice in cDMEM for 5 minutes at 350 g to remove the residual Percoll.

After blocking with purified α-FcγR (2.4G2), single cell suspensions were stained for multicolor flow cytometry. Monoclonal antibodies specific for MHC class II (10-3.6), B220 (CD45R), CD45 and CD11b were purchased from BD Biosciences. For dead cell exclusion, samples were incubated with Hoescht 33342 (HO, Calbiochem) for 5 min on ice before analysis by flow cytometry. Cell suspensions were analyzed immediately after staining to avoid maturation and/or fixation artifacts. Dead cells (HO^{hi}) and B220^{hi} cells were excluded during analysis. For microglia and CNS-macrophage analysis, samples were analyzed with LSRII and analyzed with flowjo software (FlowJo, Ashland, Oreg.).

The microglia subsets were divided into different subsets based on the expression of CD45 and CD11b. Two different cell population subsets, CD11b+CD45^{hi} (activated microglia and infiltrating macrophages) and CD11b+CD45^{lo} (resting microglia), were further analyzed for the expression profiles of microlia/monocyte activation marker MHC class II.

The results of the flow cytometry analysis indicate that treatment with exendin-4 reduced the expression level of MHC class II on both CD11b+CD45^{hi} and CD11b+CD45^{lo} cell populations in both brain and the spinal cord, at both symptom onset (i.e., 15 days after induction) and disease peak (i.e., 28 days after induction) (FIG. 7). Each of the graphs depicted in FIG. 7 show the percentage of all cells stained at the indicated fluorescence intensities. The fluorescence intensity represents the amount of MHC class II expression on the cells surface (MHC class II expression is positively correlated with fluorescence intensity).

The results of the analysis demonstrated that, by comparison to cells from vehicle-treated control EAE animals, GLP-1R agonist treatment suppressed the expression of the APC activation marker MHC class II on brain and spinal cord microglia and infiltrating macrophages at both onset and peak of the disease.

### Example 7

### Splenocytes from Exendin-4-Treated Mice are Less Pathogenic in an Adoptive Transfer Model of EAE

This example illustrates the reduced pathogenicity of splenocytes from exendin-4 treated EAE mice in an adoptive transfer model.

EAE was induced in 8- to 12-week-old SJL/J female mice (The Jackson Laboratory, Bar Harbor, Me.) by injection of 500 μg of PLPp (139-151) emulsified in CFA (Difco) and supplemented with 4 mg/ml of M. tuberculosis (Difco) on experiment day 0. Immunized animals were treated daily with either 1 mg/kg exendin-4 (n=7) or PBS (n=7). Treated animals were sacrificed on day 5 after immunization, and spleen and draining lymph nodes were collected. Cells were cultured in vitro with 20 μg/ml PLPp (139-151). After 48 hours, the cultured cells were collected and injected into naïve SJL/J animals through tail vein injection. 20 million cells were injected in each of the animals. Animals received cells from either the exendin-4 treated donors (n=7) or the control PBS treated donors (n=7).

Recipient mice were monitored daily for clinical score and body weight changes. Clinical signs of EAE were assessed according to the following scale: 0=normal; 1=limp tail; 2=moderate hind-limb weakness; 3=moderately severe hind-limb weakness (animal can still walk); 4=severe hind-limb weakness (animal can move their hind-limbs but cannot walk); 5=complete hind limb paralysis; 6=death. As shown in FIG. 8, mice receiving donor cells from the exendin-4-treated animals had significantly less severe disease that mice receiving donor cells from PBS-treated control animals. This result demonstrated that GLP-1R agonist treatment reduces the pathogenicity of splenocytes in an adoptive transfer model of EAE.

### Example 8

### Exendin-4 Reduced the Size of Secondary Lymphoid Organs in EAE-Induced Mice

This example illustrates the effect of exendin-4 on size of secondary lymphoid organs in EAE mice.

EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400ng of pertussis toxin injected i.v. MOG-induced animals were treated daily with 1 mg/kg exendin-4 (n=7), PBS (n=7) or 4 mg/kg dexamethasone (n=3) control. One group of naïve animals (n=3) was also included. On day 5 after disease induction, animals were euthanized. Spleen and inguinal lymph nodes were dissected and weighed. Lymph node weight and spleen weight and size were markedly increased in EAE animals treated with PBS (FIGS. 9A-9C). However, lymph nodes (FIG. 9A) and spleen (FIG. 9B) of exendin-4 treated EAE animals weighed significantly less than those of control animals. Similarly, the immunosuppressant dexamethasone also reduced lymph node and spleen weight in the EAE animals. The spleen of exendin-4 treated EAE animals was comparable in size to the spleen of naïve animals (FIG. 9C). In contrast, the spleen of control EAE animals was significantly enlarged (FIG. 9C). These results demonstrated that GLP-1R agonist treatment was effective in reducing the size of secondary lymphoid organs in EAE.

### Example 9

### Immunological Changes After Exendin-4 Therapy in Autoimmune Disease

This example illustrates immunological changes in EAE mice after exendin-4 treatment.

Current drugs for the treatment of MS (including corticosteroids and the CD20 antibody RITUXAN™) are immunomodulators, which exhibit immunosuppressive, T cell depleting, B cell depleting and/or other effects with respect to immune response. To determine whether GLP-1R agonists affect T cell and B-cell populations in EAE, immunological changes in EAE animals were assessed after exendin-4 treatment. For comparison, animals were treated with PBS as a negative control.

EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Animals were treated with either 1 mg/kg exendin-4 (n=3) or PBS (n=3) daily from day 0 to day 6 after immunization. Treated animals were sacrificed on day 6 after immunization, together with naïve mice that were not immunized (n=3). Spleen, lymph nodes and peripheral blood were collected. After cell dissociation and lysing of red blood cells, cells were stained with fluorophore conjugated CD4, CD8, CD19 (a B cell marker) and Ter119 antibodies (BD Biosciences). Cells were analyzed using fluorescence activated cell sorting (FACS) on a FACSAria™ cell sorter (BD Biosciences).

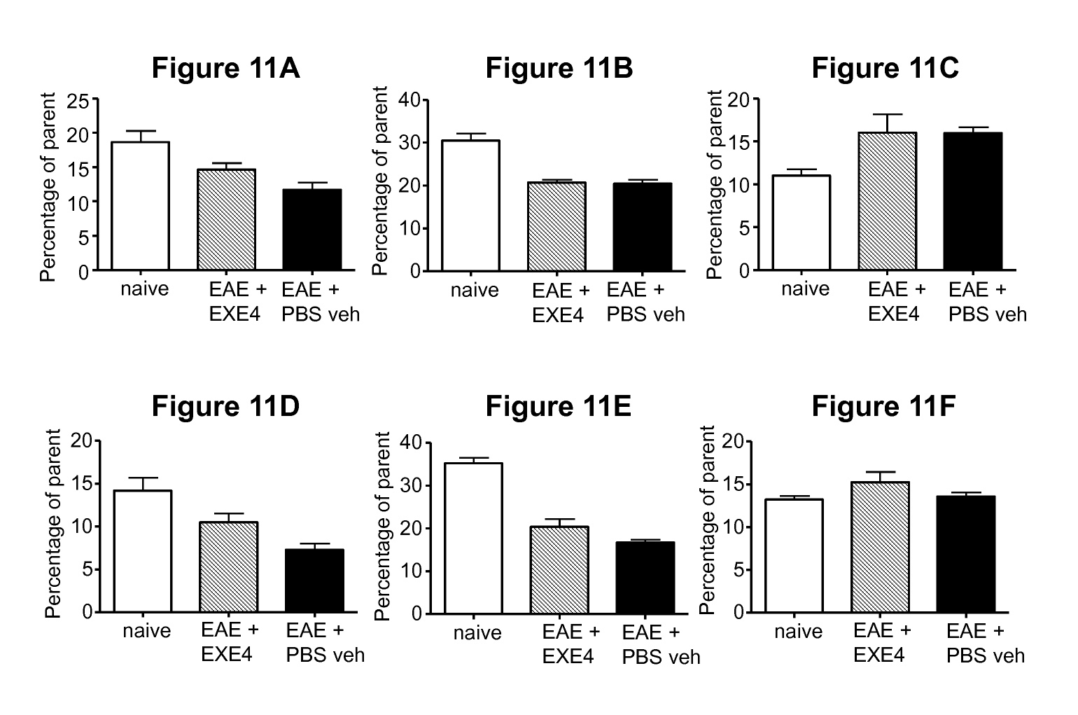

Lymphocyte levels in spleen, lymph node and blood appeared to be unchanged in exendin-4 treated EAE animals compared to PBS-treated EAE animals (FIGS. 10A-C). Monocyte levels in spleen were slightly reduced in the spleen exendin-4 treated EAE animals (FIG. 10D), but not significantly changed in lymph node or blood (FIGS. 10E and 10F). Exendin-4 treatment did not significantly change CD4+ or CD8+ cell populations (FIGS. 11A-F). The change in CD19+ population resulting from exendin-4 treatment marker just reached statistical significance (FIGS. 12A-C). These observations suggest that mechanisms of action of immunosuppressants such as corticosteroids and RITUXAN™, which directly kill CD4+, CD8+ and CD19+ cells, and GLP-1R agonists are different.

A large reduction in Ter119+ cell populations back to naïve levels in spleen was observed with exendin-4 treatment of EAE animals (FIG. 12D). Ter119+ cells are not present in lymph nodes (FIG. 12E), and are not significantly changed in blood (FIG. 12F). This result demonstrated that exendin-4 treatment was highly effective in reducing Ter119+ cell populations in spleen of EAE mice.

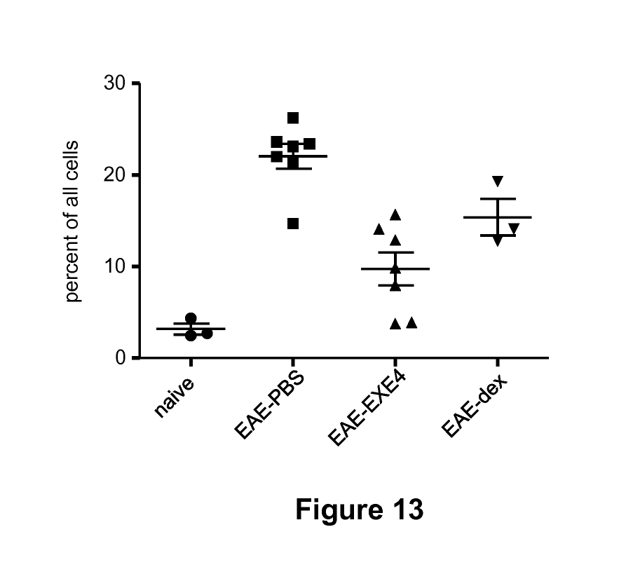

In a separate experiment, EAE was induced in female mice as described above. Animals were treated daily with 1 mg/kg exendin-4 (n=7), PBS (n=7) or 4 mg/kg dexamethasone (n=3) control. One group of naïve animals (n=3) was also included in the experiment. On day 5 after disease induction, animals were euthanized. Spleen and inguinal lymph nodes were dissected, and cells were dissociated, surfaced stained for the erythroid lineage marker Ter119 as well as CD4, and analyzed by FACS. The percentage of Ter119+ cells was determined (FIG. 13). While diseased animals had a higher percentage of Ter119+ cells compared to naïve animals, exendin-4-treated EAE animals had a reduced percentage of Ter119+ cells compared to PBS-treated EAE animals. Treatment with the immunosuppressant dexamethasone also resulted in a 20-25% reduction of Ter119+ cells in EAE animals, but not as much as with exendin-4 treatment (50˜55%). This result demonstrated that treatment with exendin-4 was effective in decreasing the percentage of Ter119+ cells in spleen of MOG immunized animals.

### Example 10

### GLP-1R Agonist Treatment Reduced T Cell Activation in Autoimmune Disease

This example illustrates changes in T cell activation levels after exendin-4 treatment of EAE mice.

EAE was induced in female C57BL/6 mice with MOG (35-55) as described above. Immunized animals were treated with PBS vehicle (n=7), 1 mg/kg exendin-4 (n=7), or 4 mg/kg dexamethasone (n=3) daily. Animals were sacrificed on day 5 after immunization, together with a naïve group of 3 mice that were not immunized. Lymph nodes were collected. After cell dissociation and lysing of red blood cells, cells were stained with fluorophore conjugated CD4 and CD44 antibodies (BDbiosciences). The cells were analyzed using a FACSaria™ cell sorter (BD Biosciences). For the analysis, FACS images were gated on CD4+ cells. Cells with high levels of CD44 (CD44^{hi}) and staining positive for CD4 were identified as activated CD4 cells. The percentage of CD4+CD44^{hi} cells was analyzed for each treatment group.

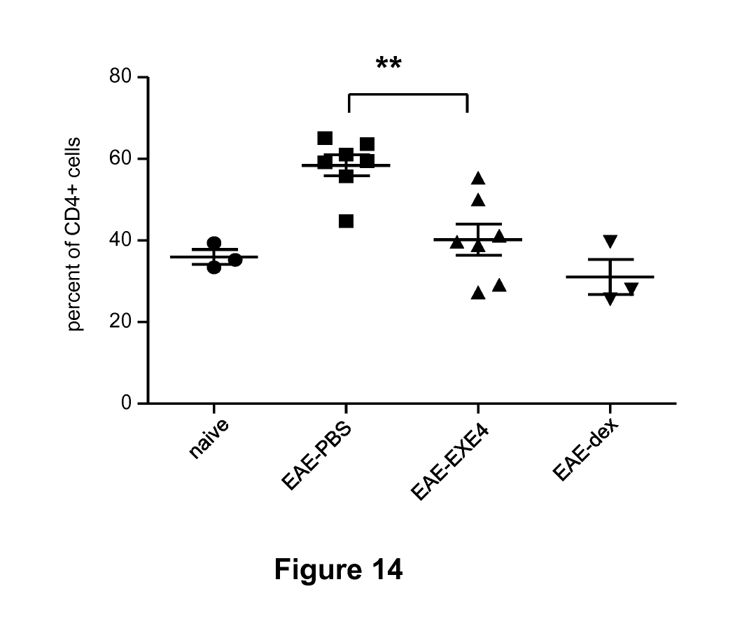

As depicted in FIG. 14, approximately 35% of CD4 cells were activated (CD4+CD44^{hi}) in naïve animals. Approximately 60% of CD4 cells from EAE animals were activated (FIG. 14). Both treatment with exendin-4 and dexamethasone reduced the percentage of activated T cells back to naïve levels (FIG. 14). Data were analyzed by one-way ANOVA followed by Dunnett's post-tests for pair-wise comparison. The two asterisks (**) indicates that the P value is less than 0.01 for the pair-wise comparison between the PBS-treated EAE and exendin-4-treated EAE groups.

This result demonstrated that treatment with exendin-4 was effective in inhibiting T cell activation after EAE induction.

### Example 11

### GLP-1R Agonist Treatment Inhibited CD4 T Cell Proliferation in Autoimmune Disease

This example illustrates changes in T cell proliferation after exendin-4 treatment of EAE mice.

EAE was induced in female C57BL/6 mice with MOG (35-55) as described above. Immunized animals were treated with PBS vehicle (n=7), 1 mg/kg exendin-4 (n=7), or 4 mg/kg dexamethasone (n=3) daily. Animals were sacrificed on day 5 after immunization, together with a naïve group of 3 mice that were not immunized. Lymph node cells were collected, cultured with MOG stimulation for 48 hours, and then treated with BrdU to identify proliferating cells. 18 hours after the addition of BrdU, cells were harvested. To measure cell proliferation, a BrdU staining kit (BD Biosciences) was used together with Pacific Blue conjugated CD4 antibodies. The cells were analyzed using a FACSaria™ cell sorter (BD Biosciences). CD4+/BrdU+ cells were identified as proliferating CD4 T cells.

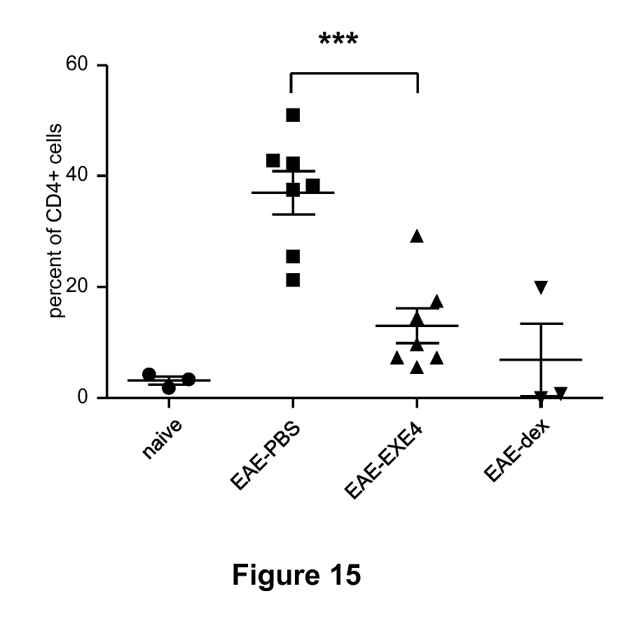

As depicted in FIG. 15, CD4 cells from naïve animals barely proliferated in response to MOG stimulation; only about 3% of CD4 T cells from naïve animals were positive for BrdU. In contrast, cells from EAE animals proliferated to a much greater extent than cells from naïve animals in response to MOG stimulation (FIG. 15). Treatment with exendin-4 and dexamesathone significantly inhibited CD4 cell proliferation (FIG. 15). Data were analyzed by one-way ANOVA followed by Dunnett's post-tests for pair-wise comparison. The three asterisks (***) indicates that the P value is less than 0.001 for the pair-wise comparison between the PBS-treated EAE and exendin-4-treated EAE groups.

This result demonstrated that treatment with exendin-4 was effective in reducing proliferation of CD4+ cells after EAE induction.

### Example 12

### GLP-1R Agonist Treatment Altered Splenocyte Cytokine Levels in Autoimmune Disease

This example illustrates changes in splenocyte cytokine levels after exendin-4 treatment of EAE mice.

To determine whether GLP-1R agonists affect cytokine levels in EAE, changes in splenocyte cytokine levels in EAE animals were assessed after exendin-4 treatment. EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Induced animals treated daily with control vehicle (PBS) or exendin-4 (1 mg/kg/day). Splenocytes (“SPL” in the first column of Table 2) and lymph node (“LN” in the first column of Table 2) cells were isolated from the treated animals on day 7 after EAE induction, and also from naïve animals (n=7). Cells were cultured 48 hours in stimulation medium containing 20 μg/ml MOG. Cytokine levels in the culture medium were measured using the Procarta® Cytokine Assay Kit (Panomics®). The kit used included bead conjugated antibodies for measurement of 32 different kinds of cytokines and chemokines.

Cytokines that showed significant change in levels between the PBS treated group (control, third column of Table 2) and exendin-4 treated group (fourth column of Table 2) are listed in Table 2. Data were analyzed by one-way ANOVA followed by Dunnett's post-tests fro pair-wise comparison. One asterisk (*) indicates that the P value is less than 0.05, and two asterisks (**) indicates that the P value is less than 0.01 for the pair-wise comparison between the PBS-treated EAE and exendin-4-treated EAE groups.

TABLE 2

| | level in naïve | level in | level in EXE4 | |
|---|---|---|---|---|
| Cytokine | animals (pg/ml) | EAE animals (pg/ml) | treated EAE animals (pg/ml) | significance |
| LN IL-17 | 1.8 ± 0.7 | 3046 ± 576 | 636.9 ± 270.4 | ** |
| LN IFN-γ | 2.4 ± 2.7 | 1297 ± 353.2 | 245.3 ± 130.4 | * |
| LN TNF-α | 4.0 ± 1.9 | 125.6 ± 23.8 | 27.9 ± 8.0 | ** |
| LN IL-3 | 1.3 ± 0.8 | 102.7 ± 27.4 | 24.9 ± 9.7 | * |
| LN SRANKL | −7.5 ± 3.1 | 63.6 ± 9.8 | 22.7 ± 4.9 | ** |
| LN mip1α | 5 ± 1.3 | 1070 ± 265.6 | 111.7 ± 46 | ** |
| LN GM-CSF | 1.4 ± 0.7 | 155 ± 45.8 | 17.7 ± 7.8 | * |
| SPL IL-23 | 28.6 ± 5.4 | 24.7 ± 5.5 | 49.1 ± 4.0 | ** |
| LN IL-23 | 14.2 ± 4.5 | 21.1 ± 2.2 | 91.4 ± 29.8 | * |
| SPL IL-12 p40 | 153.3 ± 53.3 | 259.6 ± 13.9 | 412.2 ± 57.5 | * |
| SPL IL-1a | 39.5 ± 6.9 | 27.1 ± 3.6 | 50.5 ± 2.6 | ** |
| LN BTC | 31.7 ± 3.8 | 56.8 ± 4.1 | 191.4 ± 54.8 | * |
| LN adiponectin | 139.4 ± 5.7 | 194.3 ± 13.2 | 459.3 ± 99.6 | * |

The results indicate that exendin-4 treatment reduced the level of inflammatory cytokines such as IL-17, IFN-γ, TNF-α, leading to an attenuated disease progression. The data in Table 2 are consistent with FACS analysis of cytokine expression in cells from exendin-4 treated EAE animals (see, e.g., Example 13).

### Example 13

### Exendin-4 Treatment Reduced IL-17+ and IFN-γ Cells in Lymph Node After EAE Induction

This example illustrates changes in IL-17+ and IFN-γ cell populations after exendin-4 treatment of EAE mice.

To determine whether GLP-1R agonists affect Th17 and Th1 cell populations in EAE, changes in IL-17+ and IFN-γ cell populations in EAE animals were assessed after exendin-4 treatment. IL-17 is present on Th17 cells and IFN-γ is present on Th1 cells. For comparison, animals were treated with PBS as a negative control and dexamethasone as a positive control. EAE was induced in female C57BL/6 mice by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Animals were treated daily with 1 mg/kg exendin-4 (n=7), PBS (n=7) or 4 mg/kg dexamethasone (n=3) control. One group of naïve animals (n=3) was also included. On day 5 or 7 after disease induction, animals were euthanized. Inguinal lymph nodes were dissected, cells were dissociated and analyzed by FACS. Cells were surfaced stained for CD4, and intracellularly stained for IL-17 and IFN-γ. Cells double positive for CD4 and IL-17 or CD4 and IFN-y were analyzed by FACS. While diseased animals had a higher percentage of IL-17+ cells compared to naïve animals, exendin-4-treated EAE animals had a reduced percentage of IL-17+ cells compared to PBS-treated EAE animals, both 5 and 7 days after MOG immunization (FIG. 16). Treatment with the immunosuppressant dexamethasone also resulted in a 50% reduction of IL-17+ cells in EAE animals, but not as much as with exendin-4 treatment (80-90%). This result demonstrated that treatment with exendin-4 was effective in decreasing the percentage of Ter119+ cells in lymph node.

A large reduction in IFN-γ+ cell populations in lymph node was observed with exendin-4 treatment of EAE animals (FIG. 17). As expected, the immunosuppressant dexamethasone also resulted in a reduction of IFN-γ+ cells in EAE animals. This result demonstrated that exendin-4 treatment was effective in reducing IFN-γ+ cell populations in lymph nodes of EAE mice. These results support a role for GLP-1R agonists in reducing both Th17 and Th1 cells in MOG immunized animals.

Both naturally-occurring GLP-1R agonists and GLP-1R agonist CovX bodies are effective in slowing EAE progression. The naturally-occurring agonist exendin-4 and GAC-1 were both effective in slowing disease progression. Exendin-4 was effective when administered before the onset of EAE symptoms. GAC-1 was effective when administered on a weekly basis before and after the onset of EAE symptoms. An increased initial dose of GLP-1R agonist was associated with reduced morbidity, indicating dosage-dependence. Histochemical experiments showed that GLP-1R agonists reduced invasion of CNS tissues by T-cells and monocytes. Staining for myelin showed reduced damage to nerve cells in GLP-1R agonist-treated animals. GLP-1R agonist treatment suppressed the expression of the APC activation marker MHC class II on brain and spinal cord microglia and infiltrating macrophages at both onset and peak of EAE.

Exendin-4 was used to further investigate the mechanism by which GLP-1R agonists affect EAE progression. The mechanisms of action of immunosuppressants such as corticosteroids and RITUXAN™, which directly kill CD4+, CD8+ and CD19+ cells, and GLP-1R agonists appear to differ. This was evidenced in the observation that GLP-1R agonists did not significantly change CD4+ or CD8+ cell populations in EAE-induced animals. However, GLP-1R agonists do reduce both IL-17+ and IFN-γ+ cell populations in lymph node of MOG-immunized animals. These results demonstrated that GLP-1R agonist treatment reduces Th17 and Th1 populations in lymph node.

### Example 14

### A Dipeptidyl Peptidase-4 (DPP-4) Inhibitor is Effective in Reducing Morbidity in Autoimmune Disease

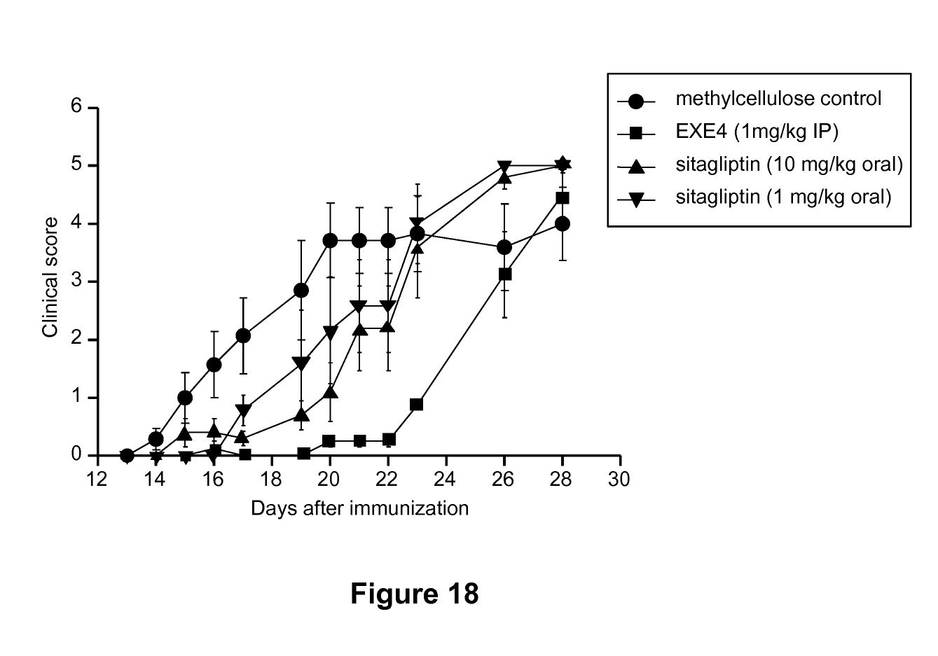

This example illustrates that elevating GLP-1 levels by treatment with a DPP-4 inhibitor can attenuate EAE progression. Animal experiments showed that the DPP-4 inhibitor sitagliptin provided significant protection against EAE morbidity compared to a methylcellulose negative control (FIG. 18).

GLP-1 is degraded in vivo by dipeptidyl peptidase-4 (DPP-4). DPP-4 inhibitors include, for example without limitation, sitagliptin, vildagliptin, saxagliptin, linagliptin, dutogliptin, gemigliptin, alogliptin and berberine. To test whether the DPP-4 inhibitor sitagliptin can prevent glucose level increases and elevated GLP-1 levels, four groups of 12 month old male C57BI/6 mice (n=4-5) were fasted overnight and subjected to an oral glucose tolerance test the following morning. One hour prior to oral glucose challenge, test animals were orally dosed with either 1 mg/kg or 10 mg/kg sitagliptin. A negative control group of animals was dosed with methylcellulose, and a positive control group was dosed with exendin-4 (1 mg/kg IP). After oral glucose challenge, glucose levels were test at 20, 40, 60 and 120 minutes. Blood samples were also taken at 20 minutes after glucose challenge to check glucose and GLP-1 levels. Exendin-4 treatment almost completely blocked glucose elevation. Treatment with either concentration of sitagliptin showed a trend toward lower glucose levels compared to the methylcellulose control, although neither reached statistical significant by two-way ANOVA. Sitagliptin at either dose significantly increased blood GLP-1 levels 20 minutes after oral glucose challenge comparing to the methylcellulose control.

To test the effect of DPP-4 inhibitor in autoimmune disease, EAE was induced in female C57BL/6 mice (8 weeks old) by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Mice were dosed once daily from day 0 to day 28 after immunization with 1 mg/kg exendin-4, 10 mg/kg sitagliptin, 1 mg/kg sitagliptin, or methylcellulose (negative control). At day 5 after immunization, blood samples were taken for measurement of GLP1 levels. Animals treated with 10 mg/kg sitagliptin had significantly higher GLP-1 levels compared with methylcellulose treated animals. To monitor disease progression, animals were scored daily for morbidity as follows: 0=normal; 1=limp tail; 2=moderate hind limb weakness; 3=moderately severe hind limb weakness (animal can still walk with difficulty); 4=severe hind limb weakness (animal can move its hind limbs but cannot walk); 5=complete hind limb paralysis; and 6=death. The animals treated with exendin-4 or sitagliptin showed significantly reduced morbidity compared to control animals (FIG. 18). As shown by the graph in FIG. 18, treatment with exendin-4 greatly attenuated EAE progression through day 25, and treatment with either 1 mg/kg or 10 mg/kg sitagliptin showed a trend towards attenuating disease symptoms. Disease score in the 10 mg/kg sitagliptin treated group was significantly lower from day 18 to day 20 compared to methylcellulose treated animals (FIG. 18). This result demonstrated that both exendin-4 and sitagliptin were effective in slowing the progression of chronic EAE.

### Example 15

### Peripheral Immune Cells are Not the Target of Exendin-4

This example illustrates that peripheral immune cells are not the target of exendin-4.

The GLP-1R agonist-antibody conjugate GAC-2 (described above) was used as a staining reagent to check expression of exendin-4/GLP-1 receptors. GAC-2 was labeled with the fluorophore Alexa488. In this example, GLP-1R expressing CHO cells were used as a positive control. GLP-1R expressing CHO cells or naïve CHO cells were blocked with 1% FBS, then stained with GAC-2-Alexa488 and analyzed by fluorescence activated cell sorting (FACS). While all cells in naïve CHO cells are negative for GAC-2 staining, 34% of GLP-1R expressing CHO cells were positive for GAC-2, indicating that GAC-2-Alexa488 can recognize GLP-1R expression on live cells.

Leukocytes from spleen, lymph node and blood from naïve animals and EAE animals were examined for expression of GLP-1R or other possible exendin-4 receptors. Both lymphocyte and monocyte macrophage compartments were analyzed for exendin-4/GLP-1 receptor expression using GAC-2. No positive signal was detected in any compartment, indicating exendin-4/GLP-1 receptors are not expressed in the peripheral lymphoid organs or their expression level is very low and below detection threshold.

To test whether exendin-4 acts directly on peripheral immune cells, lymphoid cells were treated in vitro with MOG in the presence of either exendin-4 (10 μg/ml) or vehicle (negative control). PBS instead of MOG was used as a negative control for activation. Activation of MOG-treated lymphoid cells was measured by analyzing CD44 expression (T-cell activation) and MHCII expression (APC activation). No inhibition of activation was observed when MOG-treated cells received exendin-4. These results indicate peripheral immune cells are not the target of exendin-4.

### Example 16

### Exendin-4 Treatment Reducing Morbidity in Mice with Ongoing EAE

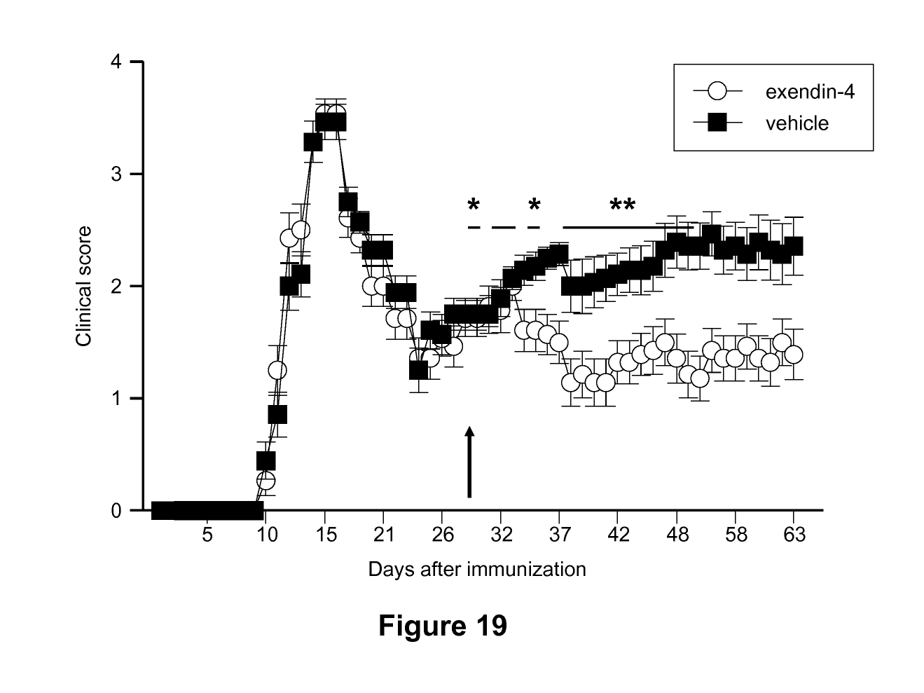

This example illustrates the efficacy of exendin-4 in treatment of ongoing EAE in mice. In experiments carried out in support of the present invention, direct administration of a GLP-1R agonist was shown to reduce morbidity in mice with ongoing EAE (FIG. 19). EAE was induced in SJL/J mice (8 to 12 weeks old, the Jackson Laboratory) by immunization with 200 μg of PLP (p139-151) dissolved in complete Freund's adjuvant (CFA) containing 4 mg/ml of heat-killed Mycobacterium tuberculosis H37Ra (Difco Laboratories) (see, Youssef et al., Nature (2002) 420:78-84). Immunized mice were examined daily for body weight measurements and clinical signs of EAE and scored. After acute EAE was established in SJL/J mice, mice were randomized into two groups based on clinical scores and body weight (n=28).

Exendin-4 treatment was started at the beginning of relapse (day 29 after immunization). Either exendin-4 (1 mg/kg) or vehicle (PBS; for control) was administered once daily (i.p.). Mice were treated daily from day 29 to day 63 after randomization. Animals were scored for morbidity as follows: 0, no paralysis; 1, loss of tail tone; 2, hindlimb weakness; 3, hindlimb paralysis; 4, hindlimb and forelimb paralysis; 5, moribund or dead. Animals treated with exendin-4 showed significantly reduced morbidity compared to control animals (FIG. 19). This result demonstrated that a GLP-1R agonist was effective in reducing morbidity in mice with ongoing EAE.

### Example 17

### Exendin-4 Treatment of Cells In Vivo Blocks Antigen-Specific CD4+T Cell Proliferation

This example illustrates inhibition of antigen-specific CD4+ T cell proliferation of cell after in vivo treatment with exendin-4.

In experiments carried out in support of the present invention, in vivo treatment of cells with a GLP-1R agonist was shown to inhibit antigen-specific CD4+ T cell proliferation (FIG. 19). Proliferation of antigen-specific CD4+ T cells from both SJL/J EAE animals and C57BL/6 EAE animals was blocked. In SJL/J female mice (8 to 12 weeks old, the Jackson Laboratory), EAE was induced by immunization with 200 μg of PLP (p139-151) dissolved in complete Freund's adjuvant (CFA) containing 4 mg/ml of heat-killed Mycobacterium tuberculosis H37Ra (Difco Laboratories) (see, Youssef et al., Nature (2002) 420:78-84). In C57BL/6 female mice, EAE was induced by s.c. injection of 200 μg of the encephalitogenic peptide MOG (35-55) emulsified in CFA (Difco) and supplemented with 4 mg/ml of M. tuberculosis (Difco) on experiment day 0, followed immediately and again after 48 hours by 400 ng of pertussis toxin injected i.v. Mice were treated daily with either exendin-4 (1 mg/kg) or vehicle only (PBS; for control).

On day 5 post-immunization, draining lymph nodes and spleens were isolated from the treated animals. Cells from the lymph nodes and spleens were washed and resuspended in pre-warmed PBS/0.1% BSA at a final concentration of 1×106 cells/ml. To monitor cell proliferation, cells were labeled with CFSE and then grown in the presence of PLP or MOG peptide. CFSE solution (Invitrogen, catalog #C34554) was added to a final concentration of 1 μM. Cells were incubated with dye at 37° C. for 10 min, then quenched by the addition of 5 volumes of ice-cold culture media and a 5 minute incubation on ice. The cells were pelleted by centrifugation and washed in fresh media, for total 3 washes. Cells were counted and incubated with either PLPp139-151 (cells from SJL/J mice) or MOGp35-55 (cells from C57BL/6 mice) peptides at 0, 10, and 50 μg/ml for 72 h. The cells were analyzed using flow cytometry. Cells lacking CFSA staining were identified as proliferating T cells. The data is summarized in Table 3.

TABLE 3

| | 72 h peptide | SJL/J EAE animals | | C57BL/6 EAE animals | |
|---|---|---|---|---|---|
| | incubation | lymph node | | lymph node | |
| Treatment | (MOG/PLP) | CD4 + T cells | spleen CD4 + T cells | CD4 + T cells | spleen CD4 + T cells |
| vehicle only | 50 μg/ml | 89.01% | 95.3% | 93.3% | 91.03% |
| | | proliferating | proliferating | proliferating | proliferating |
| vehicle only | 10 μg/ml | 87.8% | 90.01% | 93.9% | 91.6% |
| | | proliferating | proliferating | proliferating | proliferating |
| Exendin-4 | 50 μg/ml | 31.9% | 37.2% | 39.7% | 35.24% |
| | | proliferating | proliferating | proliferating | proliferating |
| Exendin-4 | 10 μg/ml | 12.8% | 15.45% | 5.05% | 9.7% |
| | | proliferating | proliferating | proliferating | proliferating |

The data in Table 3 show that exendin-4 treatment of SJL/J-EAE and C57BL/6-EAE mice resulted in a reduction of proliferating antigen-specific CD4+T cells compared to control animals. This result demonstrated that proliferation of antigen-specific CD4+T cells from EAE animals is blocked when cells are treated with a GLP-1R agonist in vivo.

In contrast to the results from in vivo exendin-4 treatment described above, exendin-4 did not affect CD4+T cell proliferation when administered to splenocytes in vitro. Briefly, CD4+T cells were isolated from naïve C57BL/6J splenocytes. To monitor cell proliferation, cells were labeled with CFSE as described above, and subsequently activated with plate-bound anti-CD3 (1 μg/ml) and anti-CD28 (0.5 pg/ml). Exendin-4 (at 0 ng/ml, 10 ng/ml, 300 ng/ml and 10 μg/ml) or vehicle (PBS) was added to the cell cultures, and the cells were treated for 72 h. The treated cells were analyzed by flow cytometry. Cells lacking CFSA staining were identified as proliferating T cells. CD4+T cells treated with exendin-4 in vitro proliferated comparably to control cells. This result demonstrated that proliferation of antigen-specific CD4+T cells is not affected when cells are treated with a GLP-1R agonist in vivo.

### Example 18

### Surface GLP-1R is Not Detected on Peripheral Immune Cells

This example illustrates the absence of exendin-4 staining in peripheral immune cells.

GLP-1R-expressing-CHO cells and parental CHO cells were incubated with FITC-labeled GAC-2 without fixation or permeabilization. Cells were incubated with primary antibody (10 μg/ml in a total of 50 μl) at room temperature for 30 minutes. The cells were analyzed using flow cytometry. FITC signal was detected on GLP-1R-expressing-CHO cells but not on the parental CHO cells. Thus, GAC-2 can detect GLP-1R on the cell surface.

Peripheral immune cells from spleen, lymph node and blood were isolated from naïve animals and EAE animals five days after immunization with MOGp35-55. The isolated cells were stained with FITC-labeled GAC-2 and analyzed by flow cytometry, gating for lymphocytes as well as monocytes. No GLP-1R staining was detected in any of these cell populations. This result suggests that these peripheral lymphoid organ cells do not express GLP-1R on their surface, or surface GLP-1R expression is below detection levels.

### Example 19

### Exendin-4 Does Not Affect MOG Activation of Immune Cells In Vitro in the 2D2 Mouse Model

This example illustrates that exendin-4 does not block in vitro MOG activation of immune cells.

Spleen and lymph node cells were isolated from seven week old 2D2 mice and cultured with or without MOG stimulation for 48 hours. Exendin-4 (10 ug/ml) or vehicle (PBS) was added to the solution of the MOG-stimulated 2D2 cell cultures. After 48 hours of MOG stimulation in culture, cells were collected, stained with anti-CD44 antibody and anti-MHC class II antibody, and analyzed by flow cytometry. Anti-CD44 antibody was used as a marker for T cell activation. Anti-MHC class II antibody was used as a marker for APC activation.

Splenocyte and lymph node cells stimulated with MOG stained positively for the presence of activated T cells. Exendin-4 treatment did not block the T cell activation. Cells cultured in the absence of MOG stimulation lacked significant staining for activated T cells. This result indicates that 2D2 splenocytes and lymph node T cells can be activated ex vivo by MOG stimulation, and that exendin-4 treatment does not block the activation directly ex vivo.

Splenocyte and lymph node cells stimulated with MOG stained positively for the presence of activated APC cells. Exendin-4 treatment did not block the APC cell activation. Cells cultured in the absence of MOG stimulation lacked significant staining for activated APC cells. This result indicates that 2D2 splenocytes and lymph node antigen presenting cells can be activated ex vivo by MOG stimulation, but exendin-4 appears not to block the activation directly ex vivo.

### Example 20

### Effect of Exendin-4 Treatment Inhibits Pro-Inflammatory Cytokine Production

This example illustrates the effect of a GLP-1R agonist on pro-inflammatory cytokine production.

The SJL/J EAE mouse model was used to investigate the effect of in vivo exendin-4 treatment on pro-inflammatory cytokine production. Relapse-remitting EAE was induced in 8 week old SJL/J animals by PLP(p139-151) immunization. One group of animals was treated with 1 mg/kg exendin-4 daily, and a second group of animals was treated with PBS (control). At day 5 after immunization, splenocytes were isolated from both groups of animals and cultured in vitro with MOG stimulation. After 48 hours of culture with MOG stimulation, the culture medium was collected and analyzed for cytokine production levels using the LUMINEX® cytokine assay for IFN-gamma and IL-17.

Splenocytes from the exendin-4-treated EAE animals produced significantly lower amounts of IFN-gamma and IL-17 compared to splenocytes from control EAE animals. This indicates that exendin-4 is efficacious in normalizing Th1 and Th17 cells after PLP(p139-151) immunization.

2D2 splenocyte cultures were used to analyze the effect of exendin-4 on pro-inflammatory cytokine production in an ex vivo system. Splenocytes from 7 week old 2D2 animals were isolated and cultured with or without MOG stimulation. In the splenocyte cultures with MOG stimulation, various concentrations of exendin-4 (10 ng/ml, 300 ng/ml and 10 ug/ml) were added to test whether exendin-4 reduces Th1 or Th17 cells ex vivo. PBS was used as a negative control. 48 hours later after the addition of exendin-4 or PBS, the culture media was collected and cytokine levels were measured. Media from cells stimulated with MOG had high levels of IFN-gamma and IL-17. In contrast, media from cells not stimulated with MOG contained minimal levels of IFN-gamma and IL-17. The presence of exendin-4 in the culture appeared to have no effect on the production of IFN-gamma. Furthermore, levels of IL-17 produced by cells treated with 10 ug/ml exendin-4 were no different from levels produced by cells not treated with exendin-4. However, media from cells treated with either 10 ng/ml or 300 ng/ml exendin-4 appeared to have slightly reduced IL-17 levels, although the difference was not statistically significant.

These results demonstrate that exendin-4 reduces pro-inflammatory cytokine production in an in vivo EAE mouse model. However, in contrast, exendin-4 did not reduce pro-inflammatory cytokine production in the 2D2 ex vivo model. These results demonstrate that exendin-4 does not appear to directly target the spleen or the lymph node.

### Example 21

### Exendin-4 Treatment Depleted CD4, CD8 Double Positive Cells in the Thymus of EAE Animals

This example illustrates the depletion of CD4, CD8 double-positive cells in the thymus of EAE animals.

To induce EAE, 8 week old C57B1/6 mice were immunized with MOG/CFA subcutaneously followed by i.p. injections of pertussis toxin immediately after immunization and 48 hours later. One group of animals were treated daily with exendin-4 (1 mg/kg; n=3), and another group were treated with control PBS (n=4). On day 4 after immunization, the thymus of the exendin-4-treated and control animals was harvested. The thymi of four naïve animals were harvested at the same time. Thymi were measured, and thymocytes were isolated, counted, stained and analyzed by flow cytometry. The data are summarized in Table 4 below.

TABLE 4

| | Thymus weight | Total thymus | % CD4, CD8 double |
|---|---|---|---|
| Mice | (g) | cell count | positive cells |
| Naive | 0.0475 ± 0.0044 | 104.6 ± 20.8 million | 84.6 ± 0.3% |
| control EAE | 0.0285 ± 0.0019 | 14.8 ± 6.5 million | 30.3 ± 11.9% |
| Exendin-4- | 0.0147 ± 0.0003 | 3.8 ± 0.8 million | 2.3 ± 0.3% |
| treated EAE | | | |

As shown in Table 4, the thymi of control EAE animals were significantly smaller compared to thymi of naïve animals (p=0.007). The thymi of exendin-4-treated EAE animals were even smaller than the thymi of the control EAE animals (p=0.002).

Thymocytes from the thymi of the control EAE animals, exendin-4 treated EAE animals and naïve animals were isolated. Consistent with the thymi measurements described above, total thymus cell counts from control EAE animals were significantly less than those of naïve animals. Total thymus cell counts from exendin-4 treated EAE animals were significantly less than those of control EAE animals.

The thymocytes were stained with anti-CD4 and anti-CD8 antibodies and analyzed by flow cytometry. 84.6±0.3% of the total thymocyte population from naïve animals were CD4, CD8 double positive. 30.3±11.9% of the total thymocyte population from control EAE animals were CD4, CD8 double positive. This data reflects the differentiation and maturation of double positive T cells in the thymus in response to MOG stimulation. In EAE animals treated with exendin-4, the CD4, CD8 double positive T cell population was almost completely abolished—only 2.3±0.3% of the total thymocyte population from exendin-4-treated EAE animals were CD4, CD8 double positive. Further studies demonstrated that there is no increase in spleen and lymph node cell counts in exendin-4-treated animals. These results suggest exendin-4 may cause depletion of CD4, CD8 double positive cells by cell death.

In a separate study, the effect of exendin-4 on T cell maturation naïve animals as well as EAE animals was investigated. EAE was induced in 8 week old C57B1/6 mice by subcutaneous injections of MOG/CFA followed by i.p. injection of pertussis toxin. The naïve and EAE animals were divided into three groups (n=3) and treated with PBS (control), exendin-4 (1 mg/kg) or dexamethasone (4 mg/kg), respectively. Dexamethasone is known to cause T cell death. On day 6 after immunization, thymi were harvested. Thymocytes were isolated, stained with anti-CD4 and anti-CD8 antibodies and analyzed by flow cytometry. The data are summarized in Table 5.

TABLE 5

| | | % CD4, CD8 |
|---|---|---|
| | | double positive |
| Mice | Treatment | cells |
| Naïve | PBS | 86.3 ± 1.1% |
| | Exendin-4 | 81.5 ± 2.0% |
| | dexamethasone | 12.4 ± 3.3% |
| EAE | PBS | 21.7 ± 6.3% |
| | Exendin-4 | 5.6 ± 2.6% |
| | dexamethasone | 1.5 ± 0.5% |

In thymocytes of EAE control animals, the CD4, CD8 double positive cell population (21.7±6.3%) was reduced compared to naïve control animals (86.3±1.1%). Both exendin-4 treatment and dexamethasone treatment acutely reduced the CD4, CD8 double positive population (5.6±2.6% and 1.5±0.5%, respectively) in EAE animals.

86.3±1.1% of the total thymocyte population from naïve animals was CD4, CD8 double positive. Dexamethasone treatment caused a significant reduction of the CD4, CD8 double positive population—only 12.4±3.3% of the total thymocyte population from dexamethasone-treated naïve animals were CD4, CD8 double positive. However, exendin-4 treatment did not affect the CD4, CD8 double positive cell population in thymi of naïve animals. 81.5±2.0% of the total thymocyte population from exendin-4-treated naïve animals were CD4, CD8 double positive.

These results demonstrate that exendin-4 treatment results in depletion of CD4, CD8 double positive cells in the thymus of EAE animals, but not naïve animals.

### Example 22

### Vagotomy Abolishes the Effect of Exendin-4 on Body Weight Loss but Does not Affect Efficacy of Exendin-4 on EAE Progression

This example illustrates the effect of vagotomy on exendin-4 treatment.

To investigate whether the effect of exendin-4 on EAE progression is mediated through the vagal nerve, EAE was induced in 8 week old vagotomized C57BI/6 animals (The Jackson Laboratory). EAE was induced in vagotomized and non-vagotomized mice by MOG/CFA subcutaneous immunization followed by i.p. injection of pertussis toxin immediately after and 48 hours later. The immunized animals were divided into two group (n=7-8). One group was treated daily with exendin-4 (1 mg/kg), and the other group was treated with PBS (control). Body weight and EAE clinical scores were monitored daily. At the end of the experiment on day 20, animals were euthanized. Stomach measurements confirmed that the vagal nerve of the vagotomized animals was properly cut.

Within the first 4 days of MOG immunization, the exendin-4-treated non-vagotomized EAE mice lost 10% of their body weight compared to control non-vagotomized EAE mice. In vagotomized mice, no difference in body weight was observed between exendin-4-treated EAE mice and control EAE mice. Thus, vagotomy abolishes the effect of exendin-4 on body weight loss. This result suggests that the effect of exendin-4 on body weight change is dependent on an intact vagal nerve.

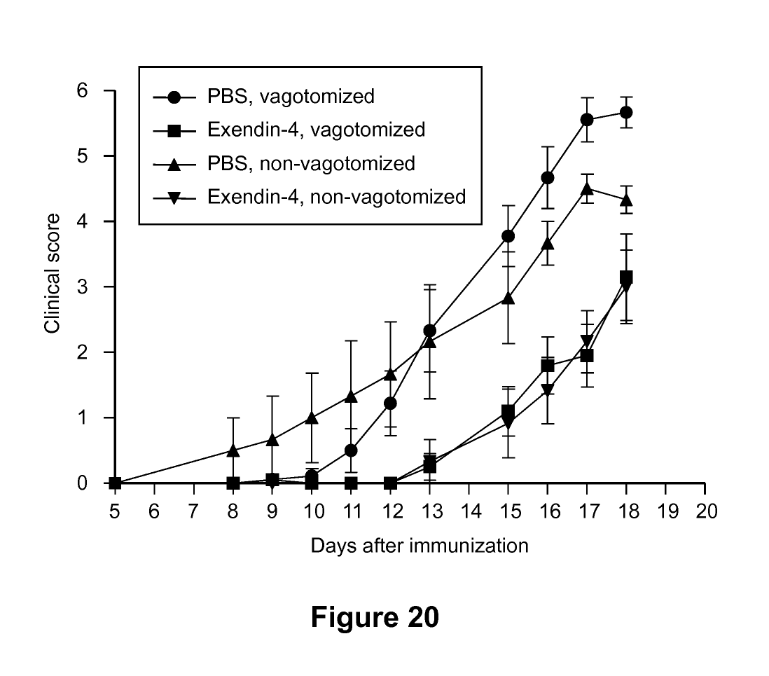

EAE disease progression was not affected by vagotomy. In both vagotomized and non-vagotomized mice, exendin-4 treatment significantly delayed EAE progression compared to PBS treated animals (FIG. 20). Thus, vagotomy does affect exendin-4-mediated delay of EAE disease progression. This result indicates that the effect of exendin-4 on EAE is not mediated by vagal nerve.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

The section headings used herein are for organizational purposes only and are not to be construed as limiting the described subject matter in any way. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method for reducing leukocyte invasion of a tissue of the central nervous system comprising administering to a mammal in need of such treatment a composition comprising a glucagon-like peptide-1 receptor (GLP-1R) agonist in an amount effective for activating GLP-1R, thereby reducing leukocyte invasion of a tissue of the central nervous system.

2. The method of claim 1, wherein the mammal has an autoimmune disorder.

3. The method of claim 2, wherein the autoimmune disorder is multiple sclerosis.

4. The method of claim 2, wherein the autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, or Graves disease.

5. The method of claim 1, wherein the mammal is a human.

6. The method of claim 1, wherein the GLP-1R agonist is OAP-189.

7. The method of claim 1, wherein the GLP-1R agonist is a DPP-4 inhibitor.

8. The method of claim 1, wherein the GLP-1R agonist is an anti-GLP-1R agonist antibody.

9. The method of claim 1, wherein the GLP-1R agonist comprises a fragment or derivative of exendin-4, wherein the fragment or derivative of exendin-4 binds to and activates GLP-1R.

10. The method of claim 1, wherein the GLP-agonist is a GLP-1R agonist-antibody conjugate (GAC) comprising a GLP-1R agonist peptide and an antibody.

11. The method of claim 10, wherein the GAC is of the structure: 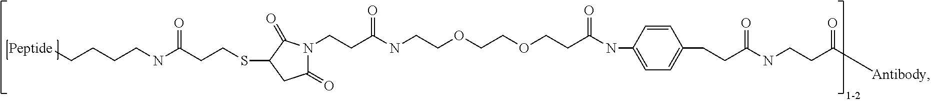
wherein the peptide is of the formula: R¹—[H¹X²E³G⁴T⁵F⁶T⁷S⁸D⁹X¹⁰S¹¹X¹²X¹³X¹⁴E¹⁵ X¹⁶X¹⁷A¹⁸X¹⁹X²⁰X²¹F²²X²³X²⁴X²⁵X²⁶X²⁷X²⁸X²⁹X³⁰X³¹X³²X³³X³⁴X³⁵X³⁶X³⁷X³⁸ X³⁹X⁴⁰ (SEQ ID NO: 3)]-R², wherein
R¹ is absent, CH₃, C(O)CH₃, C(O)CH₂CH₃, C(O)CH₂CH₂CH₃, or C(O)CH(CH₃)CH₃;
R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate, and X² is a blocking group such as Aib, A, S, T, V, L, I, D-Ala;
X¹⁰ is V, L, I, or A; X¹² is S or K;
X¹³ is Q or Y;
X¹⁴ is G, C, F, Y, W, M, or L;
X¹⁶ is K, D, E, or G;
X¹⁷ is E or Q;
X¹⁹ is L, I, V, or A;
X²⁰ is ornithine or a derivatized lysine group such as K(SH) R, or K;
X²¹ is L or E;
X²³ is I or L;
X²⁴ is A or E;
X²⁵ is W or F;
X²⁶ is L or I;
X²⁷ is I, K, or V;
X²⁸ is R, ornithine, N, or K;
X²⁹ is Aib or G;
X³⁰ is any amino acid, preferably G or R;
X³¹ is P or absent;
X³² is S or absent;
X³³ is S or absent;
X³⁴ is G or absent;
X³⁵ is A or absent;
X³⁶ is P or absent;
X³⁷ is P or absent;
X³⁸ is P or absent;
X³⁹ is S or absent; and
X⁴⁰ is a linking residue or absent;
and wherein one of X¹⁰, S¹¹, X¹², X¹³, X¹⁴, X¹⁶, X¹⁷, X¹⁹, X²⁰, X²¹, X²⁴, X²⁶, X²⁷, X²⁸, X³², X³³, X³⁴, X³⁵, X³⁶, X³⁷, X³⁸, X³⁹, or X⁴⁰ is substituted with a linking residue comprising a nucleophilic side chain covalently linked to the combining site of the antibody via a linker, wherein the linking residue is selected from the group consisting of K, R, Y, C, T, S, homologs of lysine (including K(SH)), homocysteine, and homoserine.

12. The method of claim 11, wherein the GAC comprises the structure:

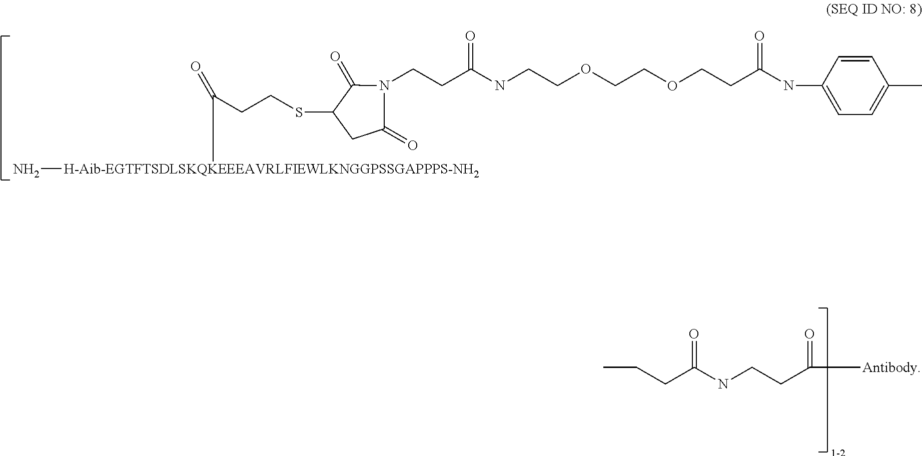

13. The method of claim 11, wherein the GAC comprises the structure:

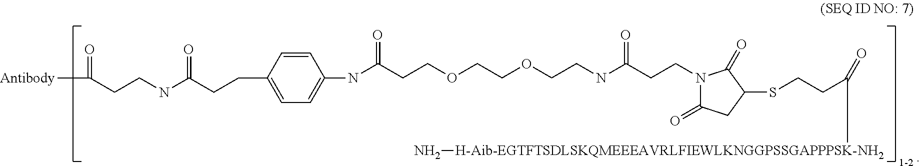

14. The method of claim 10, wherein the antibody is selected from the group consisting of a full length antibody, a Fab, a Fab′, a F(ab′)₂, an F_{c}, a dsFᵥ, an scFᵥ, a V_{H}, a diabody and a minibody.

15. The method of claim 10, wherein the antibody comprises a constant domain selected from the group consisting of IgG1, IgG2, IgG3, and IgG4.


SEQUENCE LISTING (43 sequences)

SEQ ID NO: 1 - protein, 30 aa, Homo sapiens
HAEGTFTSDVSSYLEGQAAKEFIAWLVKGR

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 40 aa, Artificial Sequence
  GLP-1 variant
HXEGTFTSDXSXXXEXXAXXXFXXXXXXXXXXXXXXXXXX

SEQ ID NO: 4 - protein, 30 aa, Artificial Sequence
  Synthetic
HXEGTFTSDVSSYLEGQAAKEFIAWLVKGR

SEQ ID NO: 5 - protein, 40 aa, Artificial Sequence
  Synthetic
HXEGTFTSDLSKQMEEEAVRLFIEWLKNGGPSSGAPPPSX

SEQ ID NO: 6 - protein, 40 aa, Artificial Sequence; identical to SEQ ID NO: 5
  Synthetic

SEQ ID NO: 7 - protein, 40 aa, Artificial Sequence
  Synthetic
HXEGTFTSDLSKQMEEEAVRLFIEWLKNGGPSSGAPPPSK

SEQ ID NO: 8 - protein, 39 aa, Artificial Sequence
  Synthetic
HXEGTFTSDLSKQKEEEAVRLFIEWLKNGGPSSGAPPPS

SEQ ID NO: 9 - protein, 31 aa, Artificial Sequence
  Synthetic
HXEGTFTSDVSSYLEGQAAKEFIAWLVKGRK

SEQ ID NO: 10 - protein, 30 aa, Artificial Sequence; identical to SEQ ID NO: 4
  Synthetic

SEQ ID NO: 11 - protein, 219 aa, Mus musculus
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, Mus musculus
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 31 aa, Artificial Sequence; identical to SEQ ID NO: 9
  Synthetic

SEQ ID NO: 14 - protein, 30 aa, Artificial Sequence
  Synthetic
HXEGTFTSDVSSYLEGQAAKEFIAWLVKXR

SEQ ID NO: 15 - protein, 31 aa, Artificial Sequence
  Synthetic
HXEGTFTSDVSSYLEGQAAKEFIAWLVKXRK

SEQ ID NO: 16 - protein, 30 aa, Artificial Sequence; identical to SEQ ID NO: 14
  Synthetic

SEQ ID NO: 17 - protein, 30 aa, Artificial Sequence; identical to SEQ ID NO: 14
  Synthetic

SEQ ID NO: 18 - protein, 30 aa, Artificial Sequence
  Synthetic
HXEGTFTSDKSSYLEEQAVKEFIAWLIKXR

SEQ ID NO: 19 - protein, 30 aa, Artificial Sequence
  Synthetic
HXEGTFTSDVSSYLEEQKVKEFIAWLIKXR

SEQ ID NO: 20 - protein, 40 aa, Artificial Sequence
  Synthetic
HXEGTFTSDVSSYLEEQAVKEFIAWLIKXRPSSGAPPPSK

SEQ ID NO: 21 - protein, 39 aa, Artificial Sequence
  Synthetic
HXEGTFTSDVSSYLEEQAVKEFIAWLIKXRPSSGAPPPS

SEQ ID NO: 22 - protein, 40 aa, Artificial Sequence
  Synthetic
HGEGTFTSDLSKQMEEEAVRLFIEWLKNGGPSSGAPPPSK

SEQ ID NO: 23 - protein, 39 aa, Artificial Sequence
  Synthetic
HXEGTFTSDLSKQMEEEAVRLFIEWLKNGGPSSGAPPKS

SEQ ID NO: 24 - protein, 39 aa, Artificial Sequence
  Synthetic
HXEGTFTSDLSKQMEEEAVRLFIEWLKNGGPSSGAKPPS

SEQ ID NO: 25 - protein, 39 aa, Artificial Sequence
  Synthetic
HXEGTFTSDLSKQMEEEAVRLFIEWLKNGGPSSKAPPPS

SEQ ID NO: 26 - protein, 39 aa, Artificial Sequence
  Synthetic
HXEGTFTSDLSKQMEEEAVRLFIEWLKNGGPKSGAPPPS

SEQ ID NO: 27 - protein, 39 aa, Artificial Sequence
  Synthetic
HXEGTFTSDLSKQMEEEAVRLFIEWLKKGGPSSGAPPPS

SEQ ID NO: 28 - protein, 39 aa, Artificial Sequence
  Synthetic
HXEGTFTSDLSKQMEEEAVRLFIEWLKNGGPSSGAPPPS

SEQ ID NO: 29 - protein, 39 aa, Artificial Sequence
  Synthetic
HXEGTFTSDLSKQMEEEAVRLFIEWKKNGGPSSGAPPPS

SEQ ID NO: 30 - protein, 39 aa, Artificial Sequence
  Synthetic
HXEGTFTSDLSKQMEEEAVRLFIKWLKNGGPSSGAPPPS

SEQ ID NO: 31 - protein, 39 aa, Artificial Sequence
  Synthetic
HXEGTFTSDLSKQMEEEAVRLFKEWLKNGGPSSGAPPPS

SEQ ID NO: 32 - protein, 39 aa, Artificial Sequence
  Synthetic
HXEGTFTSDLSKQMEEEAVRKFIEWLKNGGPSSGAPPPS

SEQ ID NO: 33 - protein, 39 aa, Artificial Sequence
  Synthetic
HXEGTFTSDLSKQMEEEAVKLFIEWLKNGGPSSGAPPPS

SEQ ID NO: 34 - protein, 39 aa, Artificial Sequence
  Synthetic
HXEGTFTSDLSKQMEEEAKRLFIEWLKNGGPSSGAPPPS

SEQ ID NO: 35 - protein, 39 aa, Artificial Sequence
  Synthetic
HXEGTFTSDLSKQMEEKAVRLFIEWLKNGGPSSGAPPPS

SEQ ID NO: 36 - protein, 39 aa, Artificial Sequence
  Synthetic
HXEGTFTSDLSKQMEKEAVRLFIEWLKNGGPSSGAPPPS

SEQ ID NO: 37 - protein, 39 aa, Artificial Sequence
  Synthetic
HXEGTFTSDLSKKMEEEAVRLFIEWLKNGGPSSGAPPPS

SEQ ID NO: 38 - protein, 39 aa, Artificial Sequence; identical to SEQ ID NO: 28
  Synthetic

SEQ ID NO: 39 - protein, 39 aa, Artificial Sequence
  Synthetic
HXEGTFTSDLKKQMEEEAVRLFIEWLKNGGPSSGAPPPS

SEQ ID NO: 40 - protein, 31 aa, Artificial Sequence
  Synthetic
HXEGTFTSDLSKQMEEEAVRLFIEWLKNGGK

SEQ ID NO: 41 - protein, 32 aa, Artificial Sequence
  Synthetic
HXEGTFTSDLSKQMEEEAVRKFIEWLKNGGPS

SEQ ID NO: 42 - protein, 30 aa, Homo sapiens; identical to SEQ ID NO: 1

SEQ ID NO: 43 - protein, 37 aa, Artificial Sequence
  OAP-189
HAQGTFTSDYSKYLEQELVKYFIQWLKNAGPSKNNIA